(19) United States
(12) Patent Application Publication
(10) Pub. No.: US 2023/0158270 A1
(43) Pub. Date: May 25, 2023

(54) COMMUNICATION DEVICES, METHODS, AND SYSTEMS

(71) Applicant: DATAFEEL INC., Omaha, NE (US)

(72) Inventors: PATRICK TYRANCE, Jr., Delray Beach, FL (US); MATTHEW LEAPER, Omaha, NE (US)

(73) Assignee: DATAFEEL INC., Omaha, NE (US)

(21) Appl. No.: 17/922,791

(22) PCT Filed: May 3, 2021
(86) PCT No.: PCT/US2021/030532

§ 371 Date: Nov. 2, 2022

Related U.S. Application Data
(60) Provisional application No. 63/019,302, filed on May 2, 2020.

(51) Int. Cl. A61M 21/00; G16H 40/20; G16H 40/67

(52) U.S. Cl. CPC A61M 21/00; G16H 40/20; G16H 40/67; A61M 2021/0022; A61M 2209/088; A61M 2230/04; A61M 2230/10; A61M 2230/40

(57) ABSTRACT

The subject matter described herein provides systems and techniques for enhancing a user’s performance. In particular, the physiological characteristics of the user can be altered toward target characteristics to bring about a particular physiological state in the user. Multiple physiological signals of the user may be sensed. Physiological characteristics indicative of a physiological state of the user may be determined. A differential between the physiological characteristics and selected target physiological characteristics may be determined. A selected energy signal associated with a correction action may be communicated to nerves associated with the user’s skin by outputting, using an energy generator, the energy signal toward the skin with one or more different energy types based on the differential. This may allow a particular targeted physiological state to be more rapidly brought about in the user.

### TECHNICAL FIELD

Aspects of the present disclosure generally relate to communication devices, methods, and systems. Particular aspects relate to wearable and implantable communication devices that are positionable adjacent physiologic tissue and communicable with the brain using nerves associated with the physiologic tissue.

### BACKGROUND

Computer screens have emerged as the most common means for person-to-computer communication. In 2015, for example, it was estimated that the average adult spends roughly 10 hours a day looking at a screen to consume information and/or communicate with others. The human eye was not designed for all this screen time, and numerous symptoms have been associated therewith. For example, eyestrain from hours of screen time may cause instances of eye irritation, dryness, fatigue, and/or blurred vision that last for extended periods of time. These problems are increasingly common, and the near constant production of new screen-oriented devices (e.g., the next iPhone®) suggests further increases.

Alternate means for person-to-computer communications may reduce the negative effects of excessive screen time. For example, the human body includes many non-optical nerves that are capable of communicating data to the brain. The skin is the largest organ in the human body and serves multiple functions including those related to temperature modulation, immuno-regulation and sensory inputs. There is a vast network of nerves highly attuned to receiving environmental data and relaying them more centrally to the brain. It is this role of the peripheral nervous system which relays environmental inputs such as the nerves associated with the skin. Further improvements are required to better leverage these and other communication capabilities of our sensory organs. Aspects of this disclosure may solve the above referenced problems, solve other known problems, and/or overcome other deficiencies in the prior art.

### SUMMARY

In general, one aspect of the subject matter described herein includes a process of enhancing a performance of a user. A plurality of physiological signals of the user may be sensed with a processing unit during a time period with one or more sensors proximate to the user. Physiological characteristics indicative of a physiological state of the user may be determined with the processing unit during the time period based on the plurality of physiological signals. Target physiological characteristics indicative of a target physiological state of the user may be selected with the processing unit during a second time period. A differential between the physiological characteristics and the target physiological characteristics may be determined with the processing unit. An energy signal associated with a corrective action performable by the user may be selected with the processing unit during the second time period to reduce the differential. The energy signal may be communicated with the processing unit to nerves associated with skin of the user during the second time period by causing an energy generator maintained against the skin to output the energy signal in a signaldirection toward the skin with one or more different energy types at an intensity proportionate to the differential until the physiological characteristics are approximate to the targetphysiological characteristics.

The plurality of physiological signals may include brainwave signals. The one or more sensors may include a brainwave sensor that is wearable by the user and adapted to output the brainwave signals responsive to activity of the user’s brain. The brainwave signals may include measurements of electrical activity produced by the user’s brain. The plurality of physiological signals may include heart signals. The one or more sensors may include a heart sensor that is wearable by the user and adaptedto output the heart signals responsive to activity of the user’s heart. The heart signals may include measurements of electrical activity produced by the user’s heart. The plurality of physiological signals may include motion signals. The one or more sensors may include a motion sensor that is wearable by the user and adaptedto output the motion signals responsive to movements of the user’s body. The heart signals may include measurements of electrical activity produced by the movements. The plurality of physiological signals may include breath signals. The one or more sensors may include a breathsensor that is wearable by the user and adaptedto output the breathsignals responsive to activity of the user’s lungs. The breath signals may include measurements of electrical activity produced by the user’s lungs.

The physiological characteristics may be determined by at least identifying a frequency or pattern of the plurality of physiological signals that corresponds to the physiological state. The target physiological characteristics may be selected by at least receiving, with the processing unit, a selection input from the user indicating the target physiological state, and retrieving, with processing unit, the target physiological characteristics from a memory associated with the user based on the selection input received from the user. The differential may be determined by at least comparing, with the processing unit, the frequency or pattern corresponding to the physiological state with a target frequency or pattern corresponding to the target physiological state. The energy signal may be selected by at least receiving, with the processing unit, the corrective action from a plurality of corrective actions based on one or more of: the target physiological characteristics, the differential, and a criterion set by the user; and selecting, with the processing unit, the energy signal from the plurality of different energy signals based on the received corrective action.

The energy generator may be operable to output a plurality of different energy types in the signal direction toward the skin. The energy generator may be caused to output the energy signal by at least selecting, with the processing unit, the one or more different energy types from the plurality of different energy types based on the energy signal. The energy generator may include a plurality of energy generators. Each energy generator of the plurality of energy generators may be operable to output a plurality of different energy types in the signal direction toward the skin. The plurality of energy generators may be caused to output the energy signal by at least selecting, with the processing unit, the one or more different energy types from the plurality of different energy types and one or more energy generators of the plurality of energy generators; and causing, with the processing unit, the one or more energy generators to output the energy signal using the one or more different energy types. Each energy generator may include a plurality of generator elements. Each generator element may be operable to output one energy type of the plurality of different energy types in the signal direction. For each energy generator, the plurality of generator elements may include one or more of an impact generator element; a heat generator element; a shock generator element; and a pressure generator element.

The energy signalmay be communicated by at least outputting the energy signalwith the one or more different energy types at a minimum intensity when the differential is within a minimum range indicating that the physiological characteristics are consistent with the target physiological characteristics; and outputting the energy signal with the one or more different energy types at a maximum intensity when the differential is within a maximum range indicating that the physiological characteristics are not consistent with the target physiological characteristics. The energy signal may be output, with the energy generator, with a first combination of the one or more different energy types when the differential is within the minimum range. The energy signal may be output, with the energy generator, with a second combination of the one or more different energy types when the differential is within the maximum range.

The plurality of physiological signals of the user may be continuously monitored, with the processing unit, during the time period with the plurality of physiological sensors. The differential at different intervals during the time period may be determined with the processing unit. The second time period may be automatically initiated, with the processing unit, by causing the energy generator to output the energy signal when the differential for a preceding interval of the different intervals is greater than a minimum trigger value.

The plurality of physiological signals of the user may be continuously monitored, with the processing unit, during the second time period with the plurality of physiological sensors. The differential at different intervals may be determined, with the processing unit, during the second time period. The energy generator may be caused to cease outputting the energy signal when the differential for a preceding interval of the second different intervals is less than a minimum trigger value for a minimum amount of time. The target physiological state may include one or more of brainwave signals indicating one of a high relaxation brain state, and a high concentration brain state; heart signals indicating one of a low pulse rate, a low blood pressure, and a high blood oxygen level; motion signals indicating one of a smooth motion rate, and a low impact motion rate; and breathsignals indicating one of a slow breathing rate, a depth of breath, and a high blood oxygen level.

### BRIEF DESCRIPTION OF THE DRAWINGS

The accompanying drawings are incorporated in and constitute a part of this specification. These drawings illustrate exemplary aspects of the present disclosure that, together with the written descriptions provided herein, serve to explain the principles of this disclosure.

FIG. 1A depicts an exemplary energy signal output onto a living tissue;

FIG. 1B depicts an exemplary communication device configured to output the energy signal of FIG. 1A;

FIG. 2A depicts a top-down view of the FIG. 1B device;

FIG. 2B depicts a bottom-up view of the FIG. 1B device;

FIG. 2C depicts a cross-section view of the FIG. 1B device taking along section line A-A of FIG. 2A;

FIG. 3A depicts a cross-section of an exemplary energy generator;

FIG. 3B depicts a bottom-up view of the FIG. 3A generator;

FIG. 4A depicts an impact energy output with the FIG. 3A generator;

FIG. 4B depicts a heat energy output with the FIG. 3A generator;

FIG. 4C depicts an electrical energy output with the FIG. 3A generator;

FIG. 4D depicts a pressure energy output with the FIG. 3A generator;

FIG. 5 depicts an exemplary processing unit;

FIG. 6 depicts a cross-section of an exemplary energy generator;

FIG. 7 depicts a bottom-up view of the FIG. 6 generator;

FIG. 8A depicts an impact energy output with the FIG. 6 generator;

FIG. 8B depicts a heat energy output with the FIG. 6 generator;

FIG. 8C depicts an electrical energy output with the FIG. 6 generator;

FIG. 8D depicts a pressure energy output with the FIG. 6 generator;

FIG. 9 depicts a cross-section of an exemplary energy generator;

FIG. 10 depicts a bottom-up view of the FIG. 9 generator;

FIG. 11 depicts a cross-section of an exemplary energy generator;

FIG. 12 depicts a bottom-up view of the FIG. 11 generator;

FIG. 13A depicts a side view of the FIG. 6 generator;

FIG. 13B depicts a side view of the FIG. 6 generator when embedded in a graspable body of a data communication device.

FIG. 14A depicts a side view of the FIG. 9 generator when embedded in a wearable body of a data communication device.

FIG. 14B depicts a back view of the FIG. 9 generator when embedded in the wearable body of FIG. 14A.

FIG. 15 depicts a side view of the FIG. 11 generator when embedded in a support body of a data communication device.

### DETAILED DESCRIPTION

Aspects of the present disclosure are now described with reference to exemplary communication devices, methods, and systems. Particular aspects reference a healthcare setting, wherein the described devices, methods, and systems may allow a single caregiver to monitor vital signals for a plurality of patients without using a screen, or at least with a reduced amount of screen time. Any references to a particular setting, such as healthcare; a particular user, such as a caregiver; particular data, such as vital signals; or particular amount of screen time, are provided for convenience and not intended to limit the present disclosure unless claimed. Accordingly, the aspects disclosed herein may be utilized for any analogous communication device, method, or system - healthcare-related or otherwise.

The terms “proximal” and “distal,” and their respective initials “P” and “D,” may be used to describe relative components and features. Proximal may refer to a position closer to a hand of user, whereas distal may refer to a position further away from said hand. With respect to a hand adjacent a living tissue, for example, proximal may refer to a position away from the tissue, whereas distal may refer to a position toward said tissue. As a further example, with respect to energy directed toward the living tissue, proximal may refer to energy directed away from the tissue and distal may refer to energy directed toward the tissue. Appending the initials P or D to a number may signify its proximal or distal location or direction. Unless claimed, these directional terms are provided for convenience and not intended to limit this disclosure.

Aspects of this disclosure may be described with reference to one or more axes. For example, an element may extend along an axis, be moved along said axis in first or second direction, and/or be rotated about said axis in a first or second direction. One axis may intersect another axis, resulting in a transverse and/or perpendicular relationship therebetween. For example, two or three perpendicular axes may intersect at an origin point to define a Cartesian coordinate system. The directional terms proximal and distal may be used with reference to any axis. One axis may be a longitudinal axis extending along a length of an element, such as a central longitudinal axis extending along the length and through a centroid of the element.

Terms such as “may,” “can,” and like variation, are intended to describe optional aspects of the present disclosure, any of which may be covered by the claims set forth below. Terms such as “comprises,” “comprising,” or like variation, are intended to describe a non-exclusive inclusion, such that a device, method, or system comprising a list of elements does not include only those elements, but may include other elements not expressly listed or inherent thereto. The term “and/or” indicates a potential combination, such that a first and/or second element may likewise be described as a first element, a second element, or a combination of the first and second elements. These potential combinations are provided as examples. Numerous other combinations are inherent to this disclosure. Unless stated otherwise, the term “exemplary” is used in the sense of “example” rather than “ideal.”

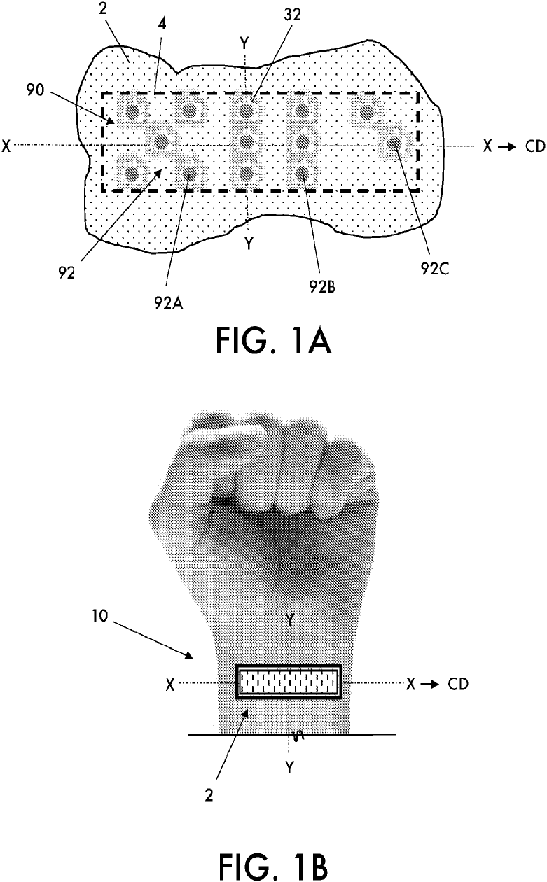

Aspects of this disclosure are directed to devices, methods, and systems for communicating with the brain through nerves associated with a living tissue. Some aspects are described with reference to an energy signal including one or more energies output to communicate symbols to the living tissue. The symbols may be used to communicate data, and the one or more energies may be used to communicate aspects of the data. The living tissue may be a portion of skin, as shown in FIGS. 1A-8D. In a healthcare setting, the energy signal may be output towards the skin of a caregiver to communicate symbols associated with a status of a patient. For example, an intensity of the one or more energies may escalate responsive to a measure of the status, providing a non-visual alert to the caregiver if the measure changes.

Exemplary energies and energy signals are now described with reference to FIG. 1A, which depicts an exemplary energy signal 90 including a plurality of symbols 92 output onto a communication area 4 of a physiologic tissue (e.g., skin 2) of user 1 with one or more different energies 32. For illustrative purposes, the symbols 92 of FIG. 1 are shown from a proximal-to-distal direction, as they would be output to the physiologic tissue (e.g., skin 2) by an energy transceiver. Each different energy 32 may be configured to communicate aspects of the data to the brain through nerves associated with the physiologic tissue (e.g., skin 2), such as nerves located distal of communication area 4.

The physiologic tissue may include skin 2 any underlying muscle, bone, and/or other portions of user 1 capable of receiving and responding to one or more different energies 32 during the second time period. For example, the one or more different energies 32 shown in FIG. 1A may be recognizable by nerves associated with skin 2 including: (i) touch receptors, such as the Meissner’s corpuscle; (ii) temperature receptors, such the Ruffini corpuscle and Krause corpuscle; (iii) electrical receptors, such as the muscles and pain receptors located in the dermis layer; (iv) pressure receptors such as the Pacinian corpuscle; and/or (v) any other cutaneous or subcutaneous nerves that innervate skin 2 or other physiologic tissues and are responsive to energies 32.

Each symbol 92 may be associated with different data. For example, in the healthcare setting, each symbol 92 may be associated with a vital sign of the patient, such as body temperature, pulse rate, respiration rate, and/or blood pressure. As shown in FIG. 1A, the plurality of symbols 92 may include a first symbol 92A, a second symbol 92B, and a third symbol 92C. In keeping with the previous example, first symbol 92A may be associated with temperature and pulse rate, second symbol 92B may be associated with respiration rate, and third symbol 92C may be associated with blood pressure. Any number of symbols 92 may be provided and/or associated with a measurable or non-measurable characteristic of the patient.

Symbols 92A, 92B, and 92C are shown as pip patterns of dots in FIG. 1A, wherein each dot is a shaded area. Each dot may represent an output of the one or more different energies 32. Aspects of energies 32 and/or each symbol 92A, 92B, and 92C may increase the complexity of energy signal 90, and thus the amount of data transmitted therewith. As shown in FIG. 1A, symbols 92A, 92B, and 92C may be scrolled across communication area 4 by outputting energies 32 toward the skin in the pip patterns; and moving the patterns across the skin in a communication direction CD. In FIG. 1A, first symbol 92A is a pip five dot pattern; second symbol 92B is a pip six dot pattern; and a third symbol 92C is a pip three dot pattern that has been truncated by an end of communication area 4 due to the scrolling. Symbols 92 may be flashed and scrolled. For example, the five dots of first symbol 92A in FIG. 1A may be output to communicate a temperature range of the patient (e.g., a normal range), and flashed on-and-off to communicate the pulse rate of the patient.

An exemplary energy transceiver 10 is depicted in FIG. 1B as being configured output energy signal 90 to communication area 4 of a physiologic tissue o fuser 1 (e.g., skin 2). As shown, energy transceiver 10 may be attached to a portion of the physiologic tissue (e.g., skin 2), including any portion located on a limb, such as the underside of a human wrist shown in FIG. 1 B for example. Communication area 4 may be sized approximate to a perimeter of transceiver 10. In this configuration, transceiver 10 may be configured to communicate energy signal 90 to the physiologic tissue (e.g., skin 2) by outputting the one or more different energies 32 toward communication area 4 in a signal direction oriented toward the physiologic tissue (e.g., skin 2). As shown in FIG. 1A, the energies 32 may be output individually and/or in combination to communicate aspects of any of symbols 92A, 92B, and 92C to the physiologic tissue (e.g., skin 2).

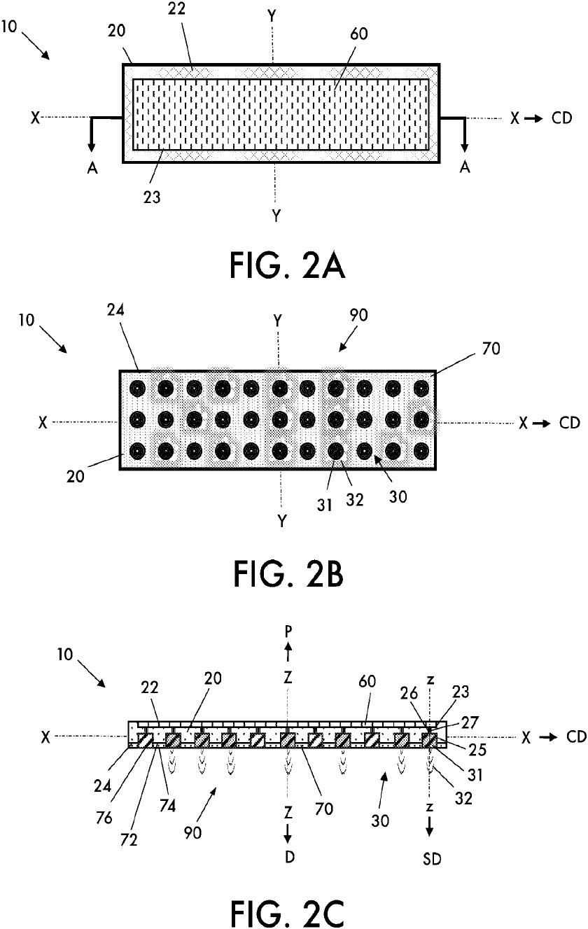

Additional aspects of exemplary energy transceiver 10 are now described with reference to FIGS. 2A-C. As shown, transceiver 10 may comprise: a body 20; a tissue interface 30; a processing unit 60; and an attachment element 70. With these elements, and the variations described herein, energy transceiver 10 may be configured to communicate energy signal 90 to nerves associated with the physiologic tissue (e.g., skin 2) by outputting the one or more different energies 32 towards the physiologic tissue with tissue interface 30.

As shown in FIGS. 2A-C, body 20 may contain the elements of energy transceiver 10. For example, body 20 of FIGS. 2A-C has a length extending along a longitudinal axis X-X, a width extending along a lateral axis Y-Y, and a thickness extending along a proximal-distal axis Z-Z. The length, width, and/or thickness of body 20 may be compatible with the physiologic tissue (e.g., skin 2). For example, body 20 may be composed of a flexible biocompatible base material, such as a polymeric material, so that the length and width of body 20 are conformable against a curvature of skin 2.

Body 20 may include any shape and be conformable with any curvature. For example, body 20 may be conformable with a cylindrical shape of a human forearm (e.g.,

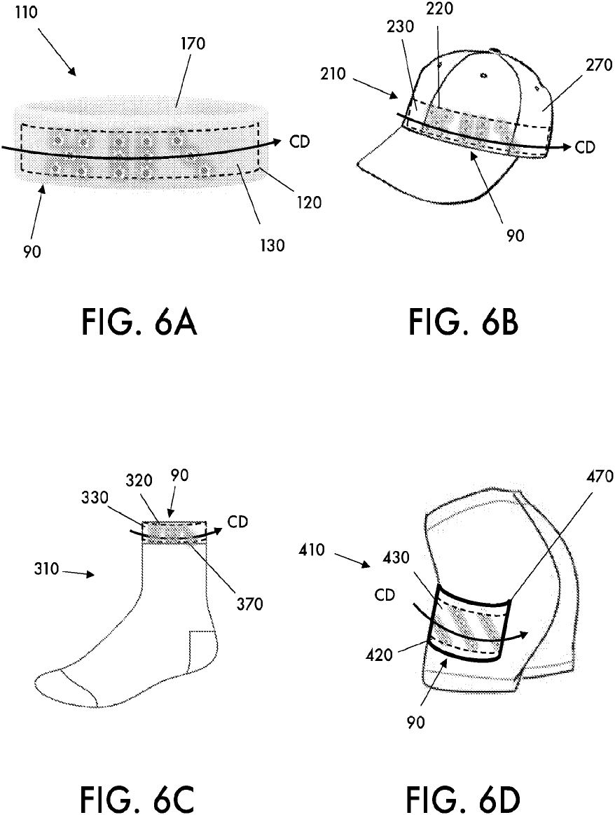

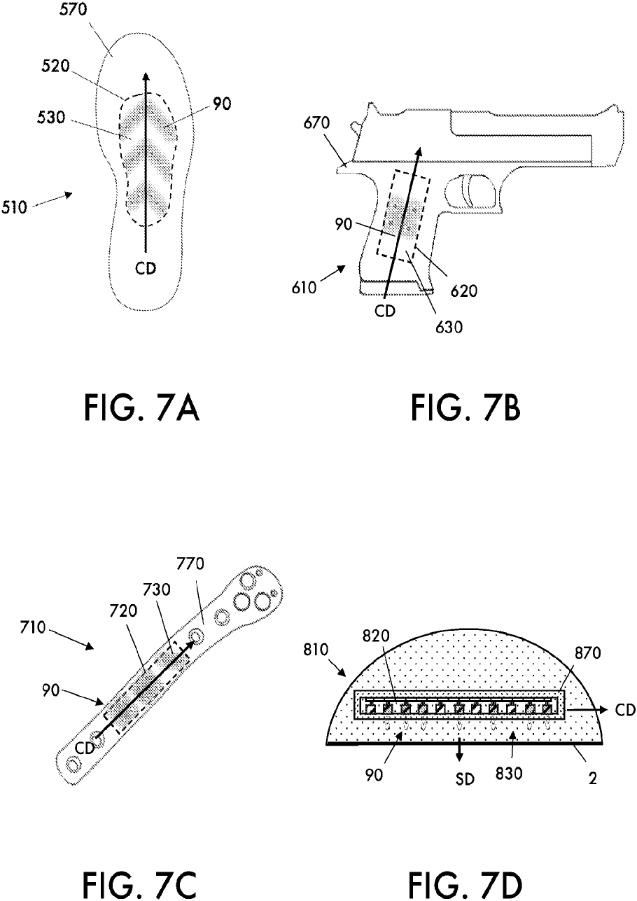

FIG. 1B) and/or comprise a semi-spherical shape a human forehead or limb (e.g., FIGS. 6A, 6B, 6C, 6D), an irregular curved shape of a human foot (e.g., FIG. 7A), an irregular curved surface of a grip (e.g., FIG. 7B), and/or surfaces of an implant (e.g., FIGS. 7C, 7D). A plurality of bodies 20 may be joined together to accommodate some curvatures. For example, side surfaces of body 20 of FIGS. 2A-C may be removable engageable with side surfaces of additional bodies 20 to create a joined layer conformable with the curvature.

The base material of body 20 may have insulating and/or energy-directing properties. For example, the base material may include compositions and/or coatings that promote energy flows along proximal-distal axis Z-Z, and limit energy flows along axes X-X and/or Y-Y. Body 20 may be manufactured from the base material using any known process. For example, body 20 may be molded or 3D printed from a base material that is biocompatible, dielectric, impact resistance, sound absorbing, and/or thermally resistant, such as polyether ether ketone (PEEK) and like polymeric materials. As a further example, body 20 may comprise any biocompatible metal (e.g., titanium) or metal alloy (e.g., stainless steel) implants or ceramic. Additional materials and/or coatings may be included with the base material and/or applied to body 20 to further promote biocompatibility.

As shown in FIGS. 2A-C, body 20 may define a proximal surface 22 (FIG. 2A) opposite of a distal surface 24 (FIG. 2B) along proximal-distal axis Z-Z (FIG. 2C). In FIGS. 2A and 2C, for example, proximal surface 22 includes a processor compartment 23 configured to receive processing unit 60. As shown, and described further below, processing unit 60 may be removable engageable (e.g., snap-fit into) with processor compartment 23. Body 20 may include and/or be compatible with additional mechanisms for securing and/or releasing the snap-fit, such as a retaining screw and/or a lever.

Body 20 of FIGS. 2A-C includes a plurality of communication bays 25. As shown, each communication bay 25 may be spaced apart from the next on distal surface 24 in a grid pattern. The spacing may be uniform or non-uniform. In FIGS. 2B and 2C, the bays 25 are spaced apart uniformly for communication with the physiologic tissue (e.g., skin 2) of FIG. 1B, which has a fairly planar surface area. Non-uniform spacing may be used to accommodate a curvature of the physiologic tissue (e.g., skin 2). As shown in FIG. 2C, each communication bay 25 may extend proximally into body 20 through distal surface 24 along a communication axis z-z that is parallel with the proximal-distal axis Z-Z of transceiver 10. In FIG. 2C, a conduit 26 extends proximally from each bay 25, through an interior portion of body 20, and into processor compartment 23, placing the plurality of bays 25 in communication with compartment 23.

Aspects of tissue interface 30 are now described with reference to FIGS. 2B and 2C. As shown, tissue interface 30 may include a plurality of energy generators 31, and each generator 31 may be located in one of communication bays 25. Each generator 31 may be operable with processing unit 60 to output energies 32 individually and/or in combination. In FIGS. 2B and 2C, for example, the one or more different energies 32 are being output from the shaded generators 31 to communicate energy signal 90 of FIG. 1A. As shown in FIG. 2C, one or more conductors 27 may extend through each conduit 26 to connect processing unit 60 to each energy generator 31, allowing control signals to be transmitted between processing unit 60 and the plurality of energy generators 31 along one or more pathways.

As shown in FIG. 2C, the one or more conductors 27 may include any number of electrical wires and/or optical fibers configured to transmit the control signals. For example, the conductors 27 may comprise a plurality of electrical conductors interconnecting the plurality of generators 31 with processing unit 60, and allowing electricity-based control signals, energies, and communications to be transmitted between unit 60 and generators 31. In addition, or alternatively, the conductors 27 may comprise a plurality of optical fibers interconnecting the plurality of generators with processing unit 60, and allowing light-based control signals, energies, and communications to be transmitted between unit 60 and generators 31. For example, each conductor 27 may comprise a twisted pair including at least one electrical conductor and at least one optical fiber. A flexible energy-insulating medium, such as an epoxy, may be used to seal conductors 27 in conduits 26.

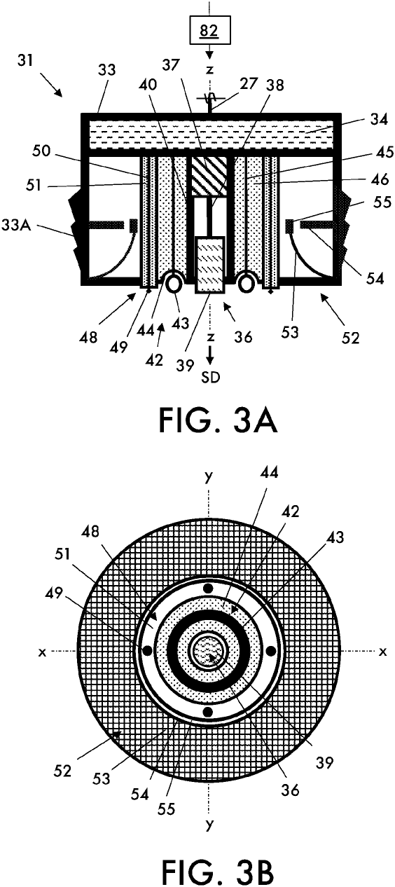

A cross-section of an exemplary energy generator 31 is depicted in FIG. 3A. As shown, each generator 31 may include: a housing 33; a controller 34; and a plurality of generator elements, such as: an impact generator element 36; a thermal generator element 42; an electrical stimulus generator element 48; and a pressure generator element 52. Examples of each generator element are now described.

Similar to body 20, housing 33 may include an insulating material that surrounds portions of each generator 31 and/or defines mounting surfaces for generator elements 36, 42, 48, and/or 52. For example, housing 33 may be made of the same base material as body 20 or a compatible material; and/or formed together with body 20 by a molding, printing, or like process. As described below, portions of each generator element 36, 42, 48, and/or 52 may extend distally from housing 33 to contact the physiologic tissue (e.g., skin 2). Housing 33 of FIG. 3A includes an attachment feature 32 configured to secure each generator 31 in one of the communication bays 25. For example, attachment feature 32 may include a set of threads on housing 33 that are engageable with an interior surface of bays 25. Other types of chemical or mechanical attachment may be used, including biocompatible adhesives, snap-fit connections, and the like.

Exemplary generator elements 36, 42, 48, and 52 may be arranged to output their respective energies 32 in approximately the same direction. As shown in FIGS. 3A and 3B, each generator element 36, 42, 48, and 52 may be arranged coaxially with communication axis z-z so that each energy 32 may be output toward the physiologic tissue (e.g., skin 2) in signal direction SD. Because of this coaxial configuration, each energy 32 may be output toward approximately the same point or area on a physiologic tissue (e.g., skin 2), making the energies 32 interchangeable. For example, any of the dots included in energy signal 90 of FIG. 1A may be interchangeably communicated to approximately the same point on skin 2 with any of the different energies 32.

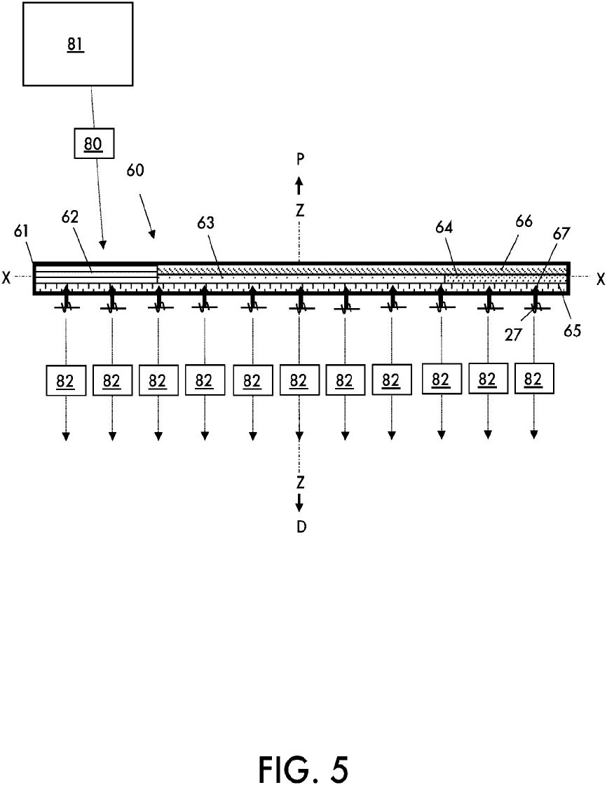

As shown in FIG. 3A, controller 34 may be configured receive a control signal 82 from processing unit 60, and activate generator elements 36, 42, 48, and 52 according to signal 82. The one or more conductors 27 may transmit the control signal 82 to generator elements 36, 42, 48, and 52 from processing unit 60 and/or direct electricity to generator elements 36, 42, 48, and 52 from a power source 66 of processing unit 60 (e.g., FIG. 5). Energy transceiver 10 may be an all-electrical device, wherein control signal 82 is an electrical signal and first and the conductors 27 are electrical wires. For varied response times, and energy requirements, transceiver 10 also may be an electro-optical device, wherein control signal 82 includes an optical signal, and at least one of the conductors 27 includes an optical fiber. For example, controller 34 may receive control signal 82 from processing unit 60 with a first one of conductors 27 (e.g., a first electrical and/or optical conductor), and direct electricity to one or more of the generator elements 36, 42, 48, and 52 with a second one of conductors 27 (e.g., a second electrical conductor) according to signal 82.

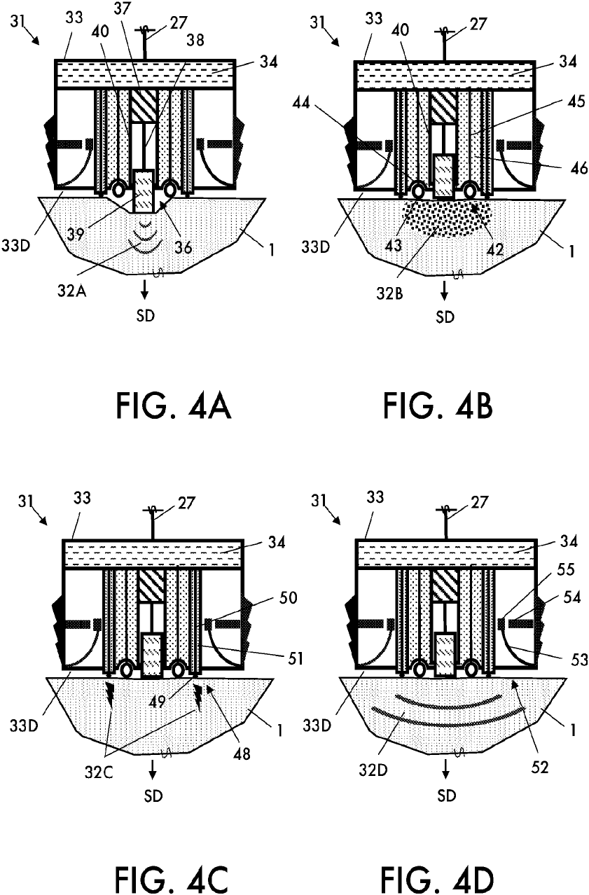

Additional aspects of generator elements 36, 42, 48, and 52 are now described with reference to FIGS. 4A-D. As shown in FIG. 4A, for example, impact generator element 36 may be configured to communicate an impact energy 32A to the brain through nerves associated with the skin 2. For example, impact generator element 36 may be a mechanical actuator that converts electricity from power source 66 into a mechanical movement recognizable by touch receptors of skin 2, such as Meissner’s corpuscle. As shown, generator element 36 may include a drive mechanism 37, a piston 38, a tissue contact 39, and a guide tube 40. Drive mechanism 37 may include a motor assembly that is attached to controller 34 and conductively engaged therewith. In this configuration, controller 34 may direct electricity to drive mechanism 37, causing the motor assembly to move piston 38 distally along communication axis z-z, outputting impact energy 32A in signal direction SD. Different force transfer components also may be used to apply energy 32A, including levers and like actuators.

As shown, drive mechanism 37 may be configured to move piston 38 between a retracted position, wherein tissue contact 39 is contained housing 33 (e.g., FIG. 3A); and an extended position, wherein at least a portion of contact 39 is distal of housing 33 (e.g., FIG. 4A). Accordingly, impact energy 32A may be output in signal direction SD as a physical movement of skin 2 caused by moving tissue contact 39 distally. Aspects of impact energy 32A may be modified. For example, outer tube 40 may be attached to housing 33 and include interior surfaces configured to modify the timing of energy 32A by guiding the proximal-distal movements of tissue contact 39 (e.g., by rotating or stabilizing contact 39). A resilient element may be added between drive mechanism 37 and contact 39 to dampen such movements.

To provide another example, impact generator element 36 also may comprise a linear resonant actuator like those sold by Precision Microdrives Limited, such as their 6 mm Linear Resonant Actuator having Model No. C12-003.001 and being available for sale at www.precisionmicrodrives.com.

Thermal generator element 42 may be configured to communicate a thermal energy 32B to the brain through nerves associated with skin 2. As shown in FIG. 4B, generator element 42 may include an electrical resistor that converts electricity from power source 66 into an amount of thermal energy recognizable by temperature receptors of skin 2 as being hot or cold, such the Ruffini corpuscle. For example, thermal generator element 42 may include an electrical resistor 43, a heat reflecting groove 44, a conductor 45, and an insulating material 46. Groove 44 may include a metal plate attached to an exterior surface of outer tube 40 of generator element 36. Resistor 33 may include an electrical wire or coil attached to groove 44. Conductor 45 may include an electrical wire extend between controller 34 and resistor 43, and material 46 may including an epoxy surrounding conductor 45.

As shown in FIG. 3B, electrical resistor 43 and heat reflecting groove 44 may be circular elements arranged coaxially with communication axis z-z. Conductor 45 may be configured to transmit electricity to electric resistor 43 for conversion into thermal energy 32B. Groove 44 may include a concave shape extending proximally into housing 33 to contain resistor 43, and the shape may include a distal surface configured to reflect heat energy 32B toward skin 2. In this configuration, thermal signal 32B may be output in signal direction SD as an amount of heat transferred to skin 2 by resistor 43. Aspects of thermal signal 32B may be modified. For example, the size, shape, and/or exterior coating of resistor 43 or groove 44 may be configured to modify the intensity of thermal energy 32B.

To provide another example, thermal generator element 42 also may comprise a flexible thermoelectric generator that utilizes the Seebeck Effect to create a temperature differential based on electric current that is perceivable by nerves associated with the physiologic tissue (e.g., skin 2), such as those sold by TEGway at www.tegway.co, making element 42 operable to cause sensations of hot and cold. Similar to as shown in FIG. 3B, the flexible thermoelectric generator may comprise an annular shape arranged coaxially with communication axis z-z.

Electrical stimulus generator element 48 may be configured to communicate an electrical energy 32C to the brain through nerves associated with skin 2. As shown in FIG. 4C, electrical stimulus generator element 48 may comprise electrodes that converts electricity from power source 66 into an electrical stimulation recognizable by electricity-sensitive receptors of the physiologic tissue, such as the muscles and pain receptors located in the dermis layer of skin 2. For example, energy generator element 48 may include at least two electric contacts 49, a conductor 50, and an insulating material 51. The conductors 50 may be metallic rods or wires extending distally from controller 34. Insulating material 51 may be an epoxy surrounding each conductor 50. Each contact 49 may include a discharge shape located on the distal-most end of one of conductors 50. In this configuration, controller 34 may direct electricity through conductors 50, and into the discharge shape of contact 49, allowing electricity to flow through skin 2 between the contacts 49 to output electrical energy 32C.

As shown in FIG. 3B, electrical contacts 49 may be spaced apart in a radial pattern coaxial with communication axis z-z. Any number of contacts 49 may be used, in any geometrical and/or spatial configuration. Insulating material 51 may be used to define and maintain the spacing. As shown, insulating materials 51 and 46 may be the same material, such as an epoxy. Four contacts 49 are shown in FIG. 3B, for example, as being arranged in two pairs. Aspects of electrical energy 32C may be modified. For example, the arrangement of contacts 49 may be changed; and/or the size of or spacing between each contact 49 changed to modify the intensity of energy 32C.

To provide another example, electrical stimulus generator element 48 may comprise any type of metal electrodes operable to apply electrical stimulation to the physiologic tissue (e.g., skin 2), such as those sold under the name Relief Band at www.reliefband.com and described in U.S. Pat. No. 7,893,761, the entirety of which is hereby incorporated by reference into this disclosure. As shown in FIG. 3C, the metal electrodes by arranged in a radial pattern surrounding the annular shape of thermal generator element 42 and arranged coaxially with communication axis z-z.

Pressure generator element 52 may be configured to communicate a pressure energy 32D to the brain through nerves associated with skin 2. As shown in FIG. 4D, pressure generator element 52 may be an electroacoustic transducer that converts electricity from power source 66 into a sound wave recognizable by pressure receptors of skin 2, such as the Pacinian corpuscle. For example, pressure generator element 52 may include a cone 53, a voice coil 54, and a magnet 55. In this configuration, controller 34 may direct electricity into voice coil 54 for interaction with magnet 55, causing movements of cone 53 that generate the pressure energy 32D in signal direction SD.

As shown in FIGS. 3B and 4D, cone 53 may have a frustoconical shape that is coaxial with communication axis z-z. An outer edge of cone 53 may be attached an interior surface of housing 33, and an inner edge of cone 53 may be attached to voice coil 54, which may be coupled to controller 34 and power source 66 by one or more conductors. As shown, coil 54 may have a circular shape, and generator elements 36, 42, and 48 may be located in the interior of said shape. Aspects of pressure energy 32D may be modified. For example, cone 53 and/or voice coil 54 may include a surround, a spider, a secondary frame, or any other structures configured to modify signal responsiveness; the strength of magnet 55 may be varied; and/or controller 34 may include an amplifier configured to modify an intensity of pressure energy 32D.

To provide another example, pressure generator element 52 also may comprise any piezoelectric ceramic speakers operable to output frequencies and sound pressures perceivable by the physiologic tissue (e.g., skin 2), such as those sold by the TDK Corporation (www.tkd.com) under the name PiezoListen™ and known to have an operating frequency range of 400 to 20,000 Hz and sound pressure of 80 dB.

Different generator element types also may be used to communicate signals to the skin with different energies 32, and/or different combinations of energies 32. For example, the plurality of generators 31 may be modified to vary individual or combined outputs of energies 32A, 32B, 32C, and 32D; and/or include additional generator elements configured to output additional signals to skin 2, including optical signals, magnetic signals, and/or any physically recognizable signals. Any type of generator element may be used and likewise coaxially arranged according to FIGS. 3A through 4D.

Additional aspects of an exemplary processing unit 60 are now described conceptually with reference to FIG. 5. Any computing technologies may be utilized. As shown in FIG. 5, processing unit 60 may be configured to receive input data 80 from a data source 81 and output control signal 82 and/or electricity to each controller 34 via conductors 27, causing activation of one or more energy generators 31. For example, processing unit 60 of FIG. 5 includes a housing 61, a data transceiver 62, one or more processors 63, a memory 64, a communication bus 65, and a power source 66.

Data source 81 may include any combination of local and/or remote data sources that are in data communication with processing unit 60. For example, source 81 may include a local sensor that is located in one of communication bays 25 and configured to send input data 80 to unit 60 using conductors 27 and/or bus 65, allowing for closed loop communications in which energy signal 90 is based on data from the local sensors. Any sensing technologies may be used. For example, the local sensor may generate the input data 80 based on chemical and/or physical outputs related to skin 2.

Data source 81 also may include a remote data source in data communication with processing unit 60 via data transceiver 62, such as a remote sensor configured to send input data 80 to processing unit 60 with data transceiver 62 over a wired or wireless connection, allowing for open loop communications in which energy signal 90 is based on data from the local sensor and/or the remote sensor.

Any number and type of local sensors may be utilized to generate input data 80. The sensor(s) may be located at any position on or relative to energy transceiver 10 where they can be in data communication with processing unit 60. In the healthcare setting, for example, one local sensor may include a personal health tracker (e.g., a Fitbit® or an iWatch®) configured to generate input data 80 based on chemical and/or physical outputs of the wearer (e.g., heart rate, temperature), and communicate input data 80 to data transceiver 62 at regular intervals (e.g., once per second or once per minute).

Housing 61 may contain the elements of processing unit 60, and/or provide a means for removing processing unit 60 from body 2, allowing for easy repairs and upgrades. As shown in FIGS. 1B and 5, for example, exterior surfaces of housing 61 may be snap-fit with interior surfaces of compartment 23 so that the distal surface of processing unit 60 is maintained against the proximal surface of compartment 23. For example, the exterior surfaces of housing 61 of may include protrusions biased outwardly along the X-X and Y-Y axes, and the interior surfaces of compartment 23 may include grooves configured to receive said protrusions.

Transceiver 62 may include any wired or wireless communication technology configured to receive input data 80 form any data source(s) 81, such as Bluetooth, Wi-Fi, and the like. As shown in FIG. 5, input data 80 may be generated with or stored on data source 81 and received with transceiver 62. In a healthcare setting, for example, data source 81 may include at least one patient monitoring device configured to send input data 80 to a remote server at regular intervals (e.g., once per minute). Data 80 may include various measures regarding the patient, such as body temperature, pulse rate, respiration rate, and/or blood pressure. For example, transceiver 62 may be configured to retrieve and/or receive data 80 from the remote server at regular intervals (e.g., once per second or once per minute).

Each control signal 82 may be received with input data 80. Data transceiver 62 may be configured to relay the signals 82 to the one or more processors 63 and/or memory 64. Alternatively, processing unit 60 may be configured to generate each control signal 82 based on input data 80. For example, memory 64 may include a signal generating program, and one more processors 63 may be configured to generate each control signal 82 with the program. In keeping with previous examples, the signal generating program may be configured to: analyze the input data 80 sent from data sources 81 including a patient monitoring device during an interval; generate symbol 92A from the temperature and pulse rate, symbol 92B from the respiration rate, and symbol 92C from the blood pressure; and output a control signal 82 for communicating the symbols 92A, 92B, and 92C to skin 2.

As shown in FIG. 5, communication bus 65 may be configured to connect the one or more processors 63 and memory 64 to each generator 31, such as to each controller 34. Bus 65 may include electrical and/or optical connectors 67 located on and/or extending distally through housing 61. For example, communication bus 65 may comprise a flexible circuit board including a proximal surface supporting elements of processing unit 60, and a distal surface including an electrical and/or optical network extending from power source 66 to the connectors 67. Any type of network may be used, such as a mesh network. Connectors 67 may be engageable with corresponding connectors of conductors 27 to provide at least one pathway for outputting control signal 82 from processing unit 60 to one or more generators 31, and/or electricity from power source 66 to one or more generators 31. Control signal 82 may include electrical and/or optical signals. For example, control signal 82 may be include a string of output commands for each generator 31, and the entire string may be output to each generator 31 utilizing the electrical and/or optical signals, adding resiliency, in which the optical signals may be utilized for faster transmission.

As described above, the snap-fit connection between housing 61 and compartment 23 may place connectors 67 in communication with conductors 27, and maintain that communication over time, allowing for continuous output of control signals 82 from processing unit 60 and/or electricity from power source 66. A cover element may be attached to the proximal surface 24 of body 20 to seal processing unit 60 within compartment 23, and/or reinforce or supplant the snap-fit connection between housing 61 and compartment 23. For example, the cover may include a graphic design, a textual element, a writing surface, and/or like decorative feature. As a further example, the cover may provide a mounting surface for other technologies, such as an antenna, signal amplifier, and/or supplemental data transceiver.

Power source 66 may include any means for supplying electricity to processing unit 60 and/or the plurality of generators 31 (e.g., to each controller 34). As shown in FIG. 5, power source 66 may include a rechargeable battery, such as a lithium-ion battery, chargeable by connection to an external power source, such as a wall outlet. Power source 66 may include power generation technologies. For example, a proximal surface of power source 66 may include a power generator, such as photovoltaic cells configured to charge the battery. As shown in FIG. 5, power source 66 also may include an optical energy source, such as a laser generator that is powered by power source 66 and configured to output optical energy to one or more generators 31 via optical pathways defined by communication bus 65 and conductors 27.

Aspects of attachment element 70 are now described with reference to FIG. 2C. As shown, attachment element 70 may be configured to maintain a position of tissue interface 30 against or adjacent skin 2. For example, element 70 may include an adhesive, elastic, and/or fastening element configured to apply a maintaining force in signal direction SD. In FIG. 2C, element 70 includes a proximal surface 72 adhered to the distal surface 24 of body 20, and a distal surface 74 adherable with skin 2. Distal surface 74 of element 70 may include a biocompatible adhesive configured to apply the maintaining force.

Attachment element 70 may be removably and/or semi-permanently attached to skin 2 by the biocompatible adhesive. For example, a first adhesive material may be used to attach the proximal surface 72 to distal surface 24, and a second adhesive material may be used to attach distal surface 74 to skin 2. As a further example, the first adhesive may be stronger so that energy transceiver 10 may be removed from skin 2 without separating surfaces 72 and 24. Either the first or second adhesive material may be biocompatible and/or may include anti-bacterial and/or moisture resistant coatings and/or compositions configured for prolonged contact with skin 2. For example, at least the second adhesive material may be configured for contact with skin 2 during the entirety of a 4-hour, 8-hour, 12-hour, 24-hour shift, or longer shift. One or both adhesives also may be configured for semi-permanent contact with skin 2, such as during the entirety of a multi-month or multi-year treatment period. For example, at least the second adhesive material may include medicinal coatings and/or compositions that promote prolonged or semi-permanent contact with skin 2 by time-releasing treatments configured to prevent or minimize contact-based injuries.

Body 20 and/or attachment element 70 may be configured to boost the efficacy of energy signal 90 by minimizing and/or maintaining the distance between tissue interface 30 and skin 2, allowing signal 90 to be communicated with less energy. For example, any of the one or more different energies 32 may be output through body 20 and/or attachment element 70. As shown in FIGS. 2B and 2C, attachment element 70 may include a plurality of openings 76. Each opening 76 may be sized approximate to one of communication bays 25, allowing the energies 32 to be output towards skin 2 in signal direction SD through openings 76. For example, each opening 76 may have an inner diameter approximate to an outer diameter of the communications bay 25 or housing 33 for each generator 31. As shown in FIG. 2C, attachment element 70 may have a thickness that allows tissue contact 39, electrical resistor 43, and/or electrical contacts 49 to contact skin 2 through opening 76 or be adjacent to skin 2 within opening 76.

Aspects of body 20 and/or attachment element 70 may direct and focus the energies 32, making it easier for the brain to distinguish one output of energies 32 from another. In keeping with previous examples, body 20 and attachment element 70 of FIGS. 2B and 2C may be composed of base materials including an impact absorbing material configured to absorb any excessive vibrations of skin 2 caused by impact energy 32A. One or both base materials may include an insulating material configured to direct thermal energy 32B, electrical energy 32C, and pressure energy 32D through openings 76 along axis Z-Z; and prevent transmission of energies 32B, 32C, and 32D along axis X-X and Y-Y. For example, body 20 and element 70 of FIG. 2C may be configured to absorb any portion of energies 32 output incidentally in directions transverse to signal direction SD to promote signal distinction by limiting unwanted communications. As a further example, each opening 76 of attachment element 70 in FIG. 2C may have a reflective coating and/or a frustoconical interior shape centered about axis z-z to further focus the energies 32 towards skin 2.

As described herein, energy transceiver 10 may be operable to communicate energy signal 90 to skin 2 by outputting any energy 32, such as impact energy 32A, thermal energy 32B, electrical energy 32C, and/or pressure energy 32D, individually or together. For example, any energies 32A-D may be used interchangeably or in combination to communicate any of the dots shown in FIG. 1A as symbols 92A, 92B, and 92C. As now described, aspects of each energy 32 may be modified to increase the complexity of signal 90, and thus the amount of data transmitted therewith. Modifiable aspects may include energy type, energy intensity, output duration, scroll rate, symbol shape, and the like.

Energy signal 90 may be communicated to skin 2 with energies 32, individually or together. In FIG. 1A, for example, each dot within first symbol 92A may be output with impact energy 32A; each dot within second symbol 92B may be output with thermal energy 32B; and each dot within third symbol 92C may be output with electrical energy 32C. The energies 32 may be combined for additional emphasis. For example, the first symbol 92A may be output with impact energy 32A in response to a baseline measure, and output with a combination of impact energy 32A and thermal energy 32B if the measure changes. The energies 32 also may be combined to enhance the penetration depth of energy signal 90. For example, first symbol 92A may be formed by first outputting pressure energy 32D to activate a portion of the nerves associated with skin 2, and second outputting thermal energy 32B to the activated nerves. Any individual dot may be similarly modified relative to any other dot.

The intensity of energies 32 may be modified for emphasis. For example, processing unit 60 may be configured to output first symbol 92A with impact energy 32A at a first intensity level in response to a baseline measure, and a second intensity level to highlight signal 92A if the measure changes. Output duration may be similarly modified. For example, the output duration of energies 32 may be instantaneous for normal measures, like a quick tap (e.g., about 100 ms); extended for abnormal measures, like a short hold (e.g., 500 ms to 1 s); or a combination thereof, as with Morse code. Scroll rate may be similarly modified. For example, symbols 92 may not be scrolled at all (i.e., a scroll rate of zero), and output duration may be used to communicate change over time by flashing symbols 92 off and or in a fixed position. As a further example, in the healthcare setting, the scroll rate may be based on an update schedule (e.g., one revolution per minute), and/or the output duration may be based on patient status (e.g., faster for more critical patients).

Symbol shape also may be modified. The plurality of symbols 92 are shown as pip pattern shapes in FIG. 1A, but any symbol shape may be used, particularly those amenable to dot-matrix representation. For example, the plurality of symbols 92 may include known Morse code, binary symbols, lines, and/or directional arrows that are scrolled across communication area 4 in communication direction CD. Alphanumeric symbols also may be communicated. For example, input data 80 may include a control signal 82 generated from a Twitter® feed, and the symbols 92 may include alphanumeric symbols for communicating the author, date, and content of each Tweet® contained in the feed. As a further example, input data 80 may include the subject and sender of an email, and the signal generating program included in memory 64 may be configured to: prioritize the email based on the sender; and generate a control signal 82 for outputting a set symbols 92 based on the subject, sender, and priority of the email. For example, first symbols 92 may be output with impact energy 32A to communicate the subject and/or sender of prioritized emails in a shorthand notation, and at least one of thermal energy 32B, electrical energy 32C, pressure energy 32D to communicate the priority level of the shorthand notation.

The resolution of tissue interface 30 may match or exceed the distinguishing capabilities of the nerves associated with skin 2. For example, in the grid formation shown in FIG. 2B, the resolution of tissue interface 30 may be measured as energy output per square inch, which may exceed the natural energy receptivity limits of the nerves associated with skin 2. As shown, the resolution of interface 30 may be relative to the spacing between each bay 25, the configuration of body 20 and/or attachment element 70, and/or the intensity of energies 32. The energy receptivity limits of skin 2 may vary by location. For example, energy transceiver 10 may be attached to a portion of skin 2 located in a highly innervated or sensitive area, such as the face, allowing even more complex symbol shapes to be communicated.

With sufficient resolution, tissue interface 30 may likewise be configured to output signal 90 to replicate image patterns and/or other sensory perceptions with energies 32, including any of the symbols described herein and even more complex interactions. As described herein, the multi-energy capabilities of energy transceiver 10 may be configured to layer energies 32 so as to communicate far more complex image patterns and/or sensory perceptions that would otherwise be possible by communicating with a single energy because of the natural receptivity limits of the nerves, and their tendency to become less receptive during prolonged exposures.

Additional aspects are now described with reference to aspects of an energy transceiver 3010 shown in FIGS. 13A, 13B, 14A, and 14B.

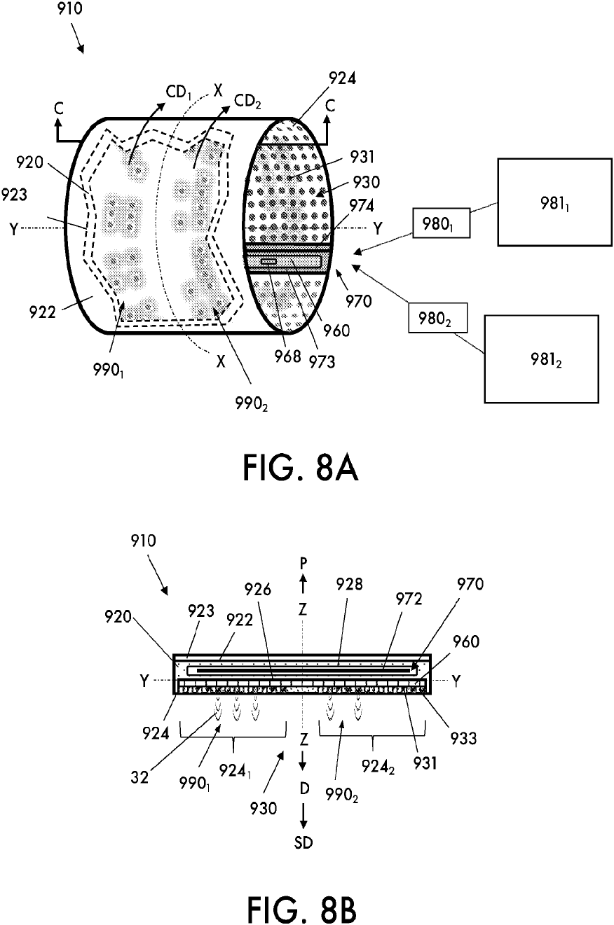

As shown in FIG. 8A, energy transceiver 3010 may include: a body 3020 and a tissue interface 3030; and an attachment element 3070, shown conceptually as a band in this example. As above, body 3020 may wrap around a circular portion of skin 2, such as around the human forearm shown in FIG. 8B. For example, as before, body 3020 may be mounted on attachment element 3070; and tissue interface 3030 may be mounted on a distal surface of body 3020, providing a curved rectangular communication area 4 and a semi-circular (e.g., less than 360°) or circular (e.g., 360°) communication direction CD for energy signal 90. In keeping with above, attachment element 3070 (e.g., the band) may be configured to maintain tissue interface 3030 against or the forearm when element 3070 is worn, allowing energy signal 90 to be output communication area 4 in signal direction SD and/or scrolled across area 4 in communication direction CD.

As described above, aspects of each energy 32 may be modified to increase the complexity of energy signal 90, and thus the amount of data transmitted therewith; and the modifiable aspects may include energy type, energy intensity, output duration, scroll rate, symbol shape, and the like, providing an incredibly broad range of obtainable complexity. Training may be required to leverage the full communicative capabilities of tissue interface 3030 and signal 90. For example, within a repetition program, the user (e.g., a person or animal) may be associatively trained to more easily and/or quickly to distinguish between: any number of known shapes output by one of energies 32, such as between a pip two dot pattern output with impact energy 32A and a pip four dot pattern output with energy 32A; or the same shape output with different energies 32, such as a pip five dot pattern with impact energy 32A or thermal energy 32B.

Communicating more complex variations, unknown signals, and/or unknown shapes may require additional training time and methods. For example, tissue interface 3030 may output energy signal 90 to include pip patterns in which each dot is output with a different combination of energies 32, allowing the pattern to be associated with a target, and each dot to be associated with a characteristic thereof. In the healthcare setting, for example, the pattern may be associated with a patient, and each dot may be associated with a different vital sign of the patient, providing immediate insight into patient health that may be updated continuously. Further training may be required to quickly distinguish between the characteristics communicated by each dot in these examples, particularly if energy signal 90 includes a plurality of pip patterns, as shown in FIG. 2C; or a dynamic shape, such as the echocardiogram depicted in FIGS. 13A and 13B; the plurality of echocardiograms depicted in FIG. 14A; and the alphanumeric symbol stream depicted in FIG. 14B as being responsive to stock market data.

Aspects of energy transceiver 3010 may be configured to provide additional communicative capabilities to, for example, assist with training. As shown in FIG. 8A, transceiver 3010 may further comprise an optical interface 3030′ compatible with eyes of the user. For example, optical interface 3030′ may comprise at least one display element operable to output an optical energy signal 90′ to the eyes, such as a flexible LCD screen or array of LEDs configured to output a plurality of colors. Any display technology may be used depending upon the power requirements of transceiver 3010. As shown in FIG. 8A, optical interface 3030′ may provide a curved optical communication area that wraps around body 3020 of transceiver 3010 along an axis X-X and/or substantially corresponds with the communication area 4. For example, tissue interface 3030 may comprise a plurality of energy generators 31 (e.g., as shown in FIGS. 3A and 3B) configured to output non-optical energy signal 90 toward skin 2 with one or more different energies 32 in a first or distal direction toward skin 2; and optical interface 3030′ may be configured to output optical signal 90′ with one or more colors in a second or proximal direction toward the eyes.

Energy transceiver 3010 may comprise a processing unit 3060 similar to any variation of processing unit 60 described herein. For example, processing unit 3060 may be operable with tissue interface 3030 and optical interface 3030′ to simultaneously communicate with nerves associated with skin 2 and the eyes by outputting signal 90 distally and signal 90′ proximally at the same time. Additional training capabilities may be realized by the simultaneous outputs. For example, the user may already be trained to react to optical signal 90′, whether or not signal 90 is communicated, such as when transceiver 3010 excludes interface 3030. Accordingly, by consistently outputting energy signal 90 with optical signal 90′, the user may be trained to react to recognize and react to energy signal 90 with or without optical signal 90′.

In a healthcare setting, for example, optical signal 90′ may communicate a vital sign of a patient to the eyes of a provider, such as the echocardiogram of FIG. 8A; and energy signal 90′ may communicate the same vital sign to skin 2 of the provider at the same time. For example, signals 90′ and 90 may be scrolled together in communication direction CD along axis X-X to simultaneously communicate aspects of the vital sign over time. As a further example, signal 90′ may comprise a plurality of colors, and the output of energies 32 in signal 90 may be modified according to a color matching algorithm to communicate similar aspects to skin 2 at the same time. Reactions to different vital signs may be trained in this manner. As shown in FIG. 8B, for example, a first portion of optical interface 3030′ may output a first optical signal 90A′, a second portion of interface 3030′ may output a second optical signal 90B′, corresponding portions of tissue interface 3030 may output corresponding energy signals 90, much like interface 930 described above. As also shown in FIG. 8B, the signals 90A′ and 90B′ may be different, in which one may be a vital sign and other may include symbols communicating related patient data as above.

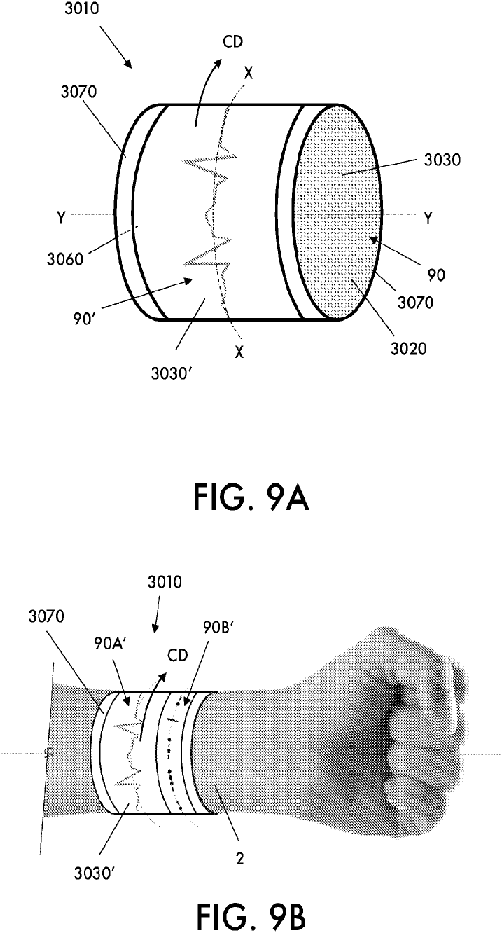

Accordingly, by simultaneously outputting optical signal 90′ together with energy signal 90, transceiver 3010 may train reactions to any stimulus, such as the exemplary vital signs and signals depicted in FIGS. 13 and 13B. As shown in FIGS. 14A and 14B, the complexity of the stimulus may be increased. For example, as shown in FIG. 9A, optical interface 3030′ and tissue interface 3030 may output their respective signals in a plurality of rows arranged around axis X-X, wherein each row includes a different set of corresponding signals movable along a communication direction CD that is transverse with axis X-X. In this example, four rows are shown as outputting four different optical signals, including a first optical signal 90A′, a second optical signal 90B′, a third optical signal 90C′, and a fourth optical signal 90D′. A corresponding set of rows and outputs may be realized by tissue interface 90.

In the healthcare setting, for example, each output of optical signals 90A′, 90B′, 90C′ and 90D′ together with its corresponding energy signal 90 may communicate a different vital sign of a different patient to a provider, training them to simultaneously monitor all of the different patients at once. As described above, aspects of each energy signal 90, such as energies 32, may be modified to communicate changes in the associated vital sign. For training purposes, the color of optical signals 90A, 90B, 90C, and 90D may be varied based on these changes so that the provider may be trained to first recognize the changes based one of the optical signals; and second recognize the same changes based on one of the energy signals based on the color matching algorithm. For example, the color matching algorithm may comprise a correspondence between visual colors and energy intensity, in which warmer colors (e.g., red) are associated with higher intensities and cooler colors (e.g., blue) are associated with lower intensities.

Another example is provided in FIG. 9B, in which each output of signals 90A′, 90B′, 90C′ and 90D′ together with its corresponding signal 90 may communicate aspects of an alphanumeric stream. As shown in FIG. 9B, for example, each alphanumeric stream may comprise a stock ticker so that the user may be trained to simultaneously monitor a plurality of tickers. As before, aspects of the different optical signals 90A′, 90B′, 90C′, and 90′D may be modified simultaneously with aspects of their corresponding energy signals 90 to communicate changes over time.

In keeping with above, optical interface 3030′ and tissue interface 3030 may be configured to individually and/or simultaneously output signals 90′ and 90 to include any symbols and shapes, as well as more complex depictions, such as graphics. For example, for more complex depictions, the color matching algorithm may be used to output different combinations of energies 32 based on color.

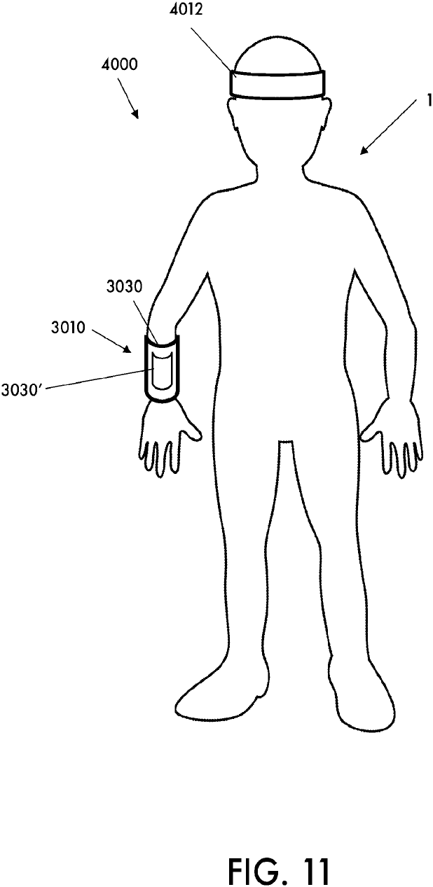

Optical interface 3030′ may comprise touchscreen capabilities allowing manipulation of signals 90 and/or 90′ by interaction therewith. For example, the position of each row depicted in FIGS. 9A and 9B may be movable via a tactile interaction with interface 3030′. As shown in FIG. 11, for example, attachment element 3070 may maintain the position of tissue interface 3030 on or adjacent skin 2 of a forearm, meaning that at least some portion of optical interface 3030′ may not be aligned with the eyes of the user at all times. Accordingly, because of the dynamic capabilities of interfaces 3030 and 3030′, the touchscreen capabilities of apparatus 3010 may allow the user to move a particular row into alignment with the eyes by scrolling the rows together around axis X-X, in which the outputs of signals 90A′, 90B′, 90C′, and 90′D and corresponding energy signal 90 move with each row. Any type of touchscreen-enabled two-way communication means may be used, including buttons, sliders, textual inputs, graphic inputs, and the like.

Any apparatus, methods, and systems described herein may be modified according to aspects of energy transceiver 3010. For example, any method steps described herein may be modified to comprise training and/or communication steps according to the above-described aspects of transceiver 3010. Aspects of each transceiver described herein may be configured to placement at a particular sensory zone of skin 2, and transceiver 3010 may be used to both tune the respective energy signals 90 for output to each zone and train the user to react accordingly based on one or more of the signals 90. The receptive capabilities of the nerves associated with skin 2 in each zone may vary, and transceiver 3010 may be configured to operate any transceivers in any system described herein so that the most complex signals are communicated to the most receptive zones.

Additional aspects in keeping with above are now described with reference to a communication system 4000. Aspects of an exemplary communication system 4000 are depicted in FIG. 1 as comprising an energy transceiver configured to receive input data and output one or more of a plurality of different energies 32 to different locations of skin 2 according to the input data. The energy transceiver may include any element and perform any function described herein with reference to any transceivers, methods, and/or systems described above. As shown in FIG. 11, for example, communication system 4000 may comprise an energy transceiver 3010 and a physiological sensor 4012.

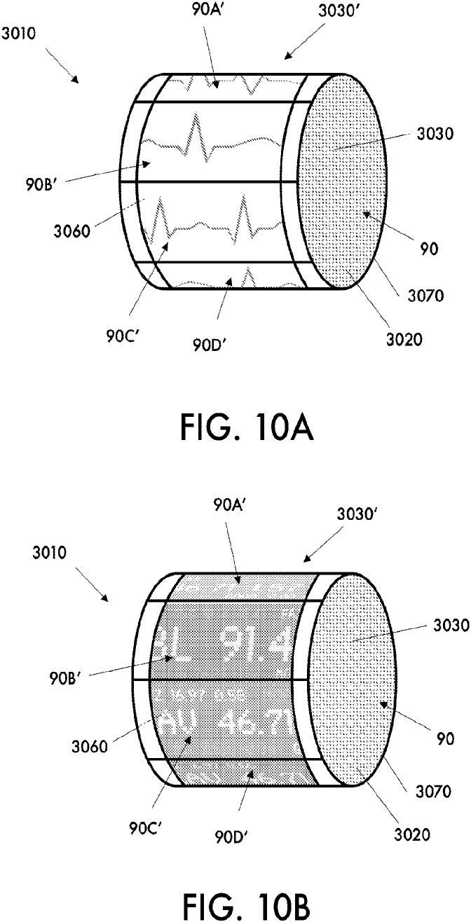

As described above and shown in FIGS. 9A, 9B, 10A, 10B, and 11, energy transceiver 3010 may comprise body 3020, tissue interface 3030, optical interface 3030′, processing unit 3060, and attachment element 3070. As shown in FIG. 11, energy transceiver 3010 may be wearable on a forearm of user 1 so that tissue interface 3030 is maintained against skin 2 of user 1 and optical interface 3030′ is oriented toward the eyes of user 1. A simplified energy transceiver 3010 also may be used in system 4000, such as one comprising a smaller number of energy generators (e.g., one or more) operable to output an energy signal in a signal direction toward skin with one or more different energies 32, without optical interface 3030′, and/or without a different attachment element 3070, including any simplified variations described herein.

As shown in FIG. 11, tissue interface 3030 may include a plurality of energy generators 31, and each generator 31 may be operable with processing unit 3060 to output different energy signals 90 with dafferent combinations of one or more different energies 32. In method 4100 described below, each energy signal 90 may comprise frequencies and/or patterns of energies 32 that user 1, has learned to associated with a different corrective action. The frequencies and/or patterns of energies 32 may be output for a limited duration of time at different intensities. In this regard, energy signal 90 may be described as an “trigger,” meaning a fairly short (e.g., ten seconds) repeatable output of frequencies and/or patterns of different energies 32 output to nerves associated with physiologic tissue (e.g., skin 2) of user 1 to help them with memory recall, much like a musical jingle output to ears of the user for the same reason.

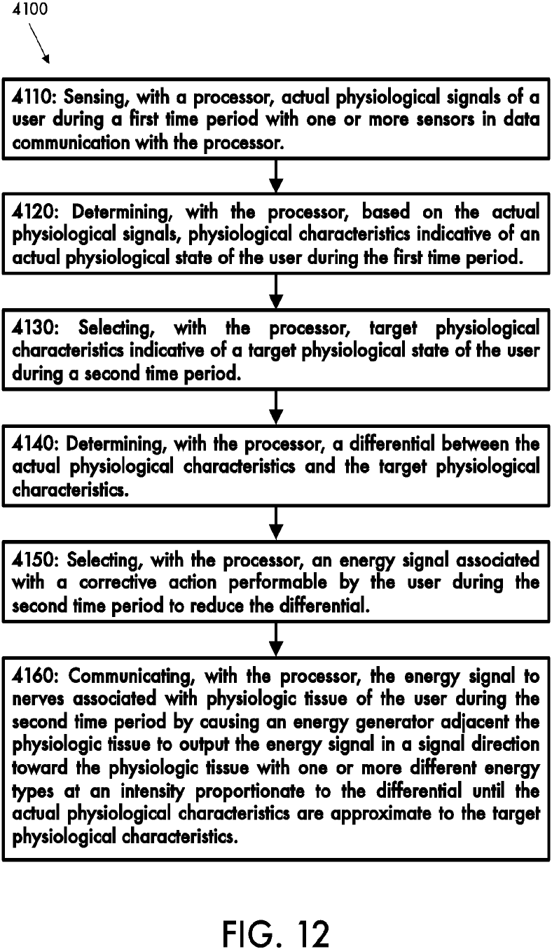

When utilized as a trigger, the intensity and/or form of energy signal 90 may be varied by processor 3060 according to method 4100 to nudge user 1 by outputting energy signal 90 (i.e., the trigger) repeatedly and with increasing intensity, until the intensity of signal 90 crosses a threshold at which it cannot physical be ignored or even causes pain in some instances, making all but impossible for user 1 not take action. For example, it is known that nerves associated with certain physiologic tissues, such as skin 2, can quickly communicate a perception of thermal energy to the brain of user 1 to help them avoid being burnt. As shown in FIG. 12 and described below, method 4100 may utilize this phenomenon to iteratively communicate with user 1 by, for example: (i) causing impact generator elements 36 of a group of energy generators 31 to output an trigger (e.g., energy signal 90) at a first intensity for a first time period, providing a subtle communication; (ii) if the differential is not reduced, further causing thermal generator elements 42 of the group of energy generators 31 to output the trigger (e.g., the same signal 90) at a second intensity for a second time period, such as first perceivable temperature that feels warm and is harder for user 1 to ignore; and (iii) if the differential is still not reduced, further causing thermal generator elements 42 of the group of energy generators 31 to output the trigger (e.g., the same signal 90) at a third intensity for a third time period, such as second perceivable temperature that feels very hot and is near impossible to ignore.

Physiological sensor 4012 may serve as a primary data source for system 4000 and methods 4100 and 4200, meaning that processing unit 3060 may input data from sensor 4012 over a network connection as part system 4000 and/or steps of methods 4100 and 4200. As shown in FIG. 11, physiological sensor 4012 may comprise one or more sensors that are adapted to measure and output physiological data about user 1, including any brain sensors and/or body sensors adapted to measure energies output directly to and/or indirectly from user 1. For example, physiological sensor 4012 may comprise any combination of known sensing technologies, such as: a brain sensor (e.g., an EEG) adapted to output brain signals for user 1; a heart sensor (e.g., PPG + Pulse Oximtery) adapted to output heart signals for user 1; a body sensor (e.g., IMU) adapted to output motion signals for user 1; and a breath sensor (e.g., PPG + Gyroscope) adapted to output breath signals for user 1. Physiologic sensor 4012 may compromise any sensing technology capable of generating measurement data associated with the body’s electrical activity such as brain (e.g., EEG), heart (e.g., ECG), muscle (e.g., EMG). Different combinations of measures such as PPG and ECG may be used to offer greater accuracy and decrease noise.

As shown in FIG. 11, physiological sensor 4012 may comprise a brainwave actuated apparatus, such as those sold under the name MUSE™ at www.choosesmuse.com and described in U.S. Pat. No. 10,582,875, and/or a wearable apparatus for brain sensors, such as that described U.S. Pat. No. 9,867,571, operable to perform methods such those described in U.S. Pat. Appn. Nos. 2015/0351 655A1, the entireties of which are incorporated by reference into this disclosure. Physiological sensor 4012 may comprise any brainwave response technologies with similar capabilities. As a further example, physiological sensor 4012 also may comprise any implantable technologies, such as those being developed under the name “Neuralink” and described at www.neuralink.com.

As shown in FIG. 12, aspects of communication system 4000 of FIG. 11 may be operable to perform a method 4100 of enhancing a performance of user 1. For example, method 4100 may comprise: (i) sensing, with processing unit 3060, actual physiological signals of user 1 during a first time period with physiological sensor 4012 (a “sensing step 4110”); (ii) determining, with processing unit 3060, based on the physiological signals, physiological characteristics indicative of an actual physiological state of user 1 during the first time period (a “determining step 4120”); (iii) selecting, with processing unit 3060, target physiological characteristics indicative of a target physiological state of user 1 during a second time period (a “selecting step 4130”); (iv) determining, with processing unit 3060, a differential between the actual physiological characteristics and the target physiological characteristics (a “determining step 4140”); (v) selecting, with processing unit 3060, an energy signal 90 associated with a corrective action performable by user 1 during the second time period to reduce the differential (a “selecting step 4150”); and (vi) communicating, with processing unit 3060, energy signal 90 to nerves associated with physiologic tissue of user 1 (e.g., skin 2) during the second time period by causing energy transceiver 3010 maintained against skin 2 to output energy signal 90 in a signal direction toward the physiologic tissue (e.g., skin 2) with one or more different energies 32 at an intensity proportionate to the differential until the physiological characteristics of user 1 are approximate to the target physiological characteristics of user 1 (a “communicating step 4160”).

Each of steps 4110-4160 may be computer implemented, meaning that are performable with processing unit 3060 and/or another processor in communication therewith according to programming for each step 4110-4160. Processing unit 3060 may comprise any computing technologies operable to perform steps 4110-4160 of method 4100 by, for example: (i) receiving input data from physiological sensor 4012 over a network; and (ii) outputting control signals over the network for causing energy transceiver 3010 to output the energy signal during the second time period responsive to the input data. In system 4000, the input data may comprise measurements of the physiological signals and any data related thereto, such as unique identifier for user 1.

Aspects of sensing step 4110 may vary according to sensing capabilities of physiological sensor 4012. For example, in step 4110, the physiological signals may comprise brainwave signals and physiological sensor 4012 may comprise a brainwave sensor adapted to output the brainwave signals responsive to activity of user 1‘s brain. The brain sensor may be wearable by user 1. As shown in FIG. 11, physiological sensor 4012 may comprise a body that is wearable on a head of user 1 so as to maintain a position of the brainwave sensor relative to the head, allowing it to sense the brainwave signals through skin 2. The brainwave signals may comprise measurements of electrical activity produced by the brain or user 1. For example, the electrical activity may indicate different ranges, low to high or slow to fast, of: (i) an information processing brain state, such as Theta waves of 3 to 8 Hz; (ii) a relaxation brain state, such as Alpha waves of 8 to 12 Hz; (iii) a concentration brain state, such as Beta waves of 12 to 36 Hz; and (iv) higher states of cognitive function, conscience, or what some call spiritual emergence, such as Gamma waves of more than 36 Hz.

In step 4110, the physiological signals also may comprise heart signals; and sensor 4012 may comprise a heart sensor adapted to output the heart signals responsive to activity of user 1’s heart. The heart sensor may be wearable by user 1. As shown in FIG. 11, the body of physiological sensor 412 may be operable to position the heart sensor relative to one or more vessels in the head of user 1, allowing sensor 412 generate heart signals based on the blood flowing through the one or more vessels. The heart signals may comprise measurements of electrical activity produced by the heart of user 1. For example, the electrical activity may indicate different ranges (e.g., low to high or slow to fast) of a pulse rate, arrhythmia, a blood pressure, a blood oxygen level, and the like.

In step 4110, the physiological signals also may comprise motion signals; and physiological sensor 4012 may comprise a motion sensor adapted to output the motion signals responsive to movements of the user 1’s body. The motion sensor may be wearable by user 1. As shown in FIG. 11, the motion sensor may comprise an inertial measurement unit or IMU mounted to the body of physiological sensor 4012, which may be operable to maintain a position of the motion sensor relative to head of user 1. The motion signals may thus comprise measurements of electrical activity output with the IMU responsive to movements of the head of user 1. For example, the electrical activity may indicate different ranges (e.g., low to high or slow to fast) of a motion rate, an impact frequency, impact intensity, and the like.

Also in step 4110, the physiological signals may comprise breath signals; and physiological sensor 4012 may comprise a breath sensor that is wearable by user 1 and adapted to output the breath signals responsive to an activity of the user 1‘s lungs. The breath sensor may be wearable by user 1. As shown in FIG. 11, the breath sensor may be mounted to the body physiological sensor 4012, which may be operable to maintain a position the breath sensor relative to the head. The breath signals may comprise measurements of electrical activity produced by the lungs of user 1. For example, the electrical activity may indicate different ranges (e.g., low to high or slow to fast) of a breathing rate, a depth of breath, a blood oxygen level, and the like.

Determining step 4120 may comprise identifying, with processing unit 3060, during the first time period, a frequency or pattern of the physiological signals that is indicative of the actual physiological state of user 1. The frequency or pattern may be identified using any actual measured values of the brain signals, heart signals, motion signals, and/or breath signals sensed during step 4110. Each actual measured value may comprise a range associated with the electrical activities described above, including ranges (e.g., low to high or slow to fast) for the relaxation brain state, concentration brain state, pulse rate, arrhythmia, blood pressure, blood oxygen level, motion rate, impact frequency, impact intensity, breathing rate, depth of breath, and the like, any of which may be used to identify the frequency or pattern.

To provide a particular example, physiological sensor 4012 may output brainwave signals including different types of brainwaves associated with different frequencies, including Theta waves (3 to 8 Hz), Alpha waves (8 to 12 Hz), Beta waves (12 to 36 Hz), and Gamma waves (> 36 Hz) and/or different patterns thereof, including different transitions therebetween, such as an Alpha-Theta transition. Brainwave signal data output by physiological sensor 4012 may define an objectively identifiable signature of user 1‘s brain and historical progression of the same that may be indicative of a physiological state of user 1 during the time period. In step 4120, processing unit 3060 may use the signature and/or historical progression to identify a probable physiological state of user 1 by comparing the signature and/or historical progression to a labelled data set of previously identified signatures and historical progressions for user 1.

To generate the labelled data set, for example, step 4120 may be preceded by a training method comprising linking, with processing unit 3060, different frequencies or patterns of the physiological signals for user 1 with different confirmed physiological states for user 1. For example, an exemplary training method may comprise: outputting to the eyes of user 1, with processing unit 3060 (e.g., via optical interface 3030′), a training stimulus adapted to invoke a physiological response from user 1 during a training period; sensing, with processing unit 3060, a physiological signals of user 1 during the training period with physiological sensor 4012; determining, with processing unit 3060, based on the physiological signals sensed by physiological sensor 4012, physiological characteristics indicative of an induced physiological state of user 1 when exposed to the training stimulus; receiving, with processing unit 3060 (e.g., via optical interface 3030′), inputs from user 1 confirming that they experienced the induced physical state indicated by the physiological characteristics; establishing, with processing unit 3060, a link between a frequency or pattern of the physiological signals and the confirmed physical state; and/or generating, with processing unit 3060, a labelled data set of previously identified signatures for user 1 comprising a listing of each confirmed physical state together with any frequencies or patterns linked thereto

Selecting step 4130 may be performed manually or automatically. For example, step 4130 may comprise: (i) receiving, with processing unit 3060, a selection input from user 1 indicating the target physiological state (e.g., via optical interface 3030′); and (ii) retrieving, with processing unit 3060, the target physiological characteristics based on the selection input received from user 1. The target physiological characteristics may be retrieved from a memory associated with user 1 based on the selection input received from the user. In step 4130, user 1 may provide the selection input by selecting a target physiological state that they would like to obtain or avoid in the near future. For example, if user 1 is an athlete, then they may wish to obtain and/or practice obtaining a high concentration physiological state often referred to as being in “the zone,” and may select that option with optical interface 3330′, causing the selection data to be sent to processing unit 3060.

As a further example, if user 1 has anger management issues, then they may wish to ovoid and/or practicing avoiding a highly agitated physiological state at which they are less likely to make good decisions; and thus, may select that option with optical interface 3330′, similarly causing that selection data to be sent to processing unit 3060. Other options may be similarly selected with optical interface 3330′ to cause different selected data to be sent to processing unit 3060 for inducing, maintaining, or disrupting different brain states, such as a relaxation brain state for rest (e.g., between 8 to 12 Hz), a concentration state for focused mental activity (12 to 36 Hz), and increased consciousness or spiritual emergence (> 36 Hz).

Selecting step 4130 may be performed automatically by: (i) receiving, with processing unit 3060 (e.g., via optical interface 3030′), a trigger criteria input; (ii) continuously monitoring, with processing unit 3060, the physiological signals output from physiological sensor 4012 during the time period; (iii) automatically selecting, with processing unit 3060, the target physiological state when a frequency or pattern of the plurality physiological signals corresponds with a frequency or pattern associated with the trigger input criteria; and (iv) retrieving, with processing unit 3060, the target physiological characteristics of the target physiological state for user 1. User 1 may utilize optical interface 3030′ to enter the trigger criteria input so that energy transceiver 4010 may be used help them obtain and/or avoid a target mental state that they are likely experience in the near future. The trigger criteria input may identify particular frequencies or patterns (e.g., of brainwaves, pulse rates, and/or breathing rate) indicative of different stages of an angry mental state for user 1. For example, step 4130 may comprise: continuously monitoring for the particular frequencies or patterns associated with the angry mental state for user 1; selecting a target physical state with a new frequency or pattern that is different or opposite of those particular frequencies or patterns; and retrieving the target physiological characteristics associated with the new frequency or pattern.

In step 4130, retrieving the target physiological characteristics of the target physiological state for user 1 may comprise retrieving, with processing unit 3060, target values for any actual physiological signals sensed by physiological sensor 4012. For example, step 4130 may comprise retrieving target values for any of the brain signals, heart signals, motion signals, and/or breath signals described above. As a further example, each target value may comprise target ranges associated with the electrical activities described above, including any combination of target ranges (e.g., low to high or slow to fast) for the relaxation brain state, concentration brain state, pulse rate, arrhythmia, blood pressure, blood oxygen level, motion rate, smoothness of the motion rate, impact motion rate, impact frequency, impact intensity, breathing rate, and/or depth of breath described above.

Determining step 4140 may comprise determining the differential between the actual physiological characteristics and the target physiological characteristics by comparing, with processing unit 3060, a frequency or pattern corresponding to the physiological state determined in step 4120 with a target frequency or pattern corresponding to the target physiological state selected in step 4130. In keeping with above, the target frequency or pattern may be defined using any target values of the brain signals, heart signals, motion signals, and/or breath signals described above. As before, each measured value may similarly comprise a target range associated with the electrical activities described above, including target ranges (e.g., low to high or slow to fast) for the relaxation brain state, concentration brain state, pulse rate, arrhythmia, blood pressure, blood oxygen level, motion rate, smoothness of the motion rate, impact motion rate, impact frequency, impact intensity, breathing rate, depth of breath, and the like, any of which may be used to define the target frequency or pattern.

For example, the different frequencies and/or patterns of user 1‘s brainwaves actually measured by physiological sensor 4012 during the first time period may define an objectively identifiable “signature” including a brain stress measurement and a brain concentration measurement. To facilitate comparison, a relational database of the different frequencies and/or patterns of user 1‘s brain when in the targeted physiological state may include counterpart values, such as a targeted brain stress measurement and a targeted brain concentration measurement. Step 4140 may thus comprise determining the differential based on mathematical differences between the respective brain stress and concentration measurements during the first time period and the respective targeted brain stress and concentration measurements.

According to these examples, each energy signal 90 output in communicating step 4160 may serve as a trigger for a corrective action that is selected by or for user 1 in step 4150 and performable by user 1 during the second time period (e.g., during step 4160) to reduce the differential determined in step 4140. Energy signals 90 output in communicating step 4160 cannot force user 1 to take the correction action, but they can remind user 1 in a nagging and/or increasingly persistent manner that becomes more obvious (e.g., painful, if needed) to user 1 relative to a measure of urgency associated with the correction action, such as the differential determined in step 4140.

Selecting step 4150 may be performed to facilitate selection of an energy signal 90 that has been previously associated with one or more corrective actions. Step 4150 may be performed manually or automatically. For example, step 4150 may comprise: (i) receiving, with processing unit 3060 (e.g., via optical interface 3030′), a selection input from user 1 indicating a corrective action associated the target physiological state; and (ii) selecting, with processing unit 3060, an energy signal 90 from a library of different energy signals 90 based on the selection input. The received corrective action may be one of multiple corrective actions based on one or more of the target physiological characteristics, the differential, and a criterion set by user 1. In this example, user 1 may have previously associated different energy signals 90 with different corrective actions by conducting training exercises with communication system 4000 that help user 1 to establish and learn associations between each energy signal 90 and a particular corrective action, making it more likely that the energy signal 90 will trigger (or compel) user 1 to take the particular corrective action. Each different energy signal 90 may thus be output with a particular combination of one or more different energies 32 to remind and/or compel user 1 to take a particular correction action and/or sequence of corrective actions.

Any type of corrective actions may be selected in step 4150, limited only by user 1‘s ability to recognize energy signal 90 and execute the corrective action responsively thereto. For example, user 1 may work with their coach and/or therapist to practice and memorize a breathing exercise (e.g., square breathing) that has been proven, over time, to help them transition from one mental state (e.g., an unfocused and/or angry state) to another, more desirable mental state (e.g., a more focused and/or less angry state); and selecting step 4150 may comprise selecting, with processing unit 3060, an energy signal 90 previously associated with the breathing exercise, allowing it to serve as a trigger for compelling user 1 to stop what they are doing and perform the breathing exercise. As a further example, step 4150 also may help user 1 execute the correction action by further selecting, with processing unit 3060, a guiding stimulus (e.g., a video) operable to guide user 1 through the corrective action and/or provide real-time feedback based on outputs from physiological sensor 4012.

Selecting step 4150 may be performed automatically by: (i) receiving, with processing unit 3060 (e.g., via optical interface 3030′), a signal criteria input; and (ii) selecting, with processing unit 3060, energy signal 90 from the library of different energy signals 90 based the signal criteria input. For example, the signal criteria input may be input with optical interface 3030′ to comprise an indication of whether user 1 is in a low intensity stimulus environment (e.g., a library) or a high intensity stimulus environment (e.g., an emergency ward or a trading floor); and step 4150 may comprise selecting, with processing unit 3060, a particular energy signal 90 that is more likely to be interpreted by user 1 in the indicated environment. As a further example, user 1 may utilize optical interface 3030′ to enter the signal criteria when transitioning from one environment to another, allowing for a customizable user experience.

As shown in FIG. 11, communicating step 4160 may provide a means for triggering user 1 by utilizing tissue interface 3030 of transceiver 3010 to communicate energy signal 90 to nerves associated with physiologic tissue (e.g., skin 2) of user 1. The communication in step 4160 may be discrete. For example, when body 3020 of transceiver 3010 is maintained against the skin (e.g., by attachment element 3070), each energy signal 90 may be output to the physiologic tissue (e.g., to skin 2) in a non-visual and/or non-audible form, making it possible to trigger or compel user 1 to take a corrective action without alerting anyone in proximity thereto, something that is much harder to do with screen-based technologies like the Apple iPhone and iWatch.

Different types of hardware may be used to output different energy signals 90. For example, tissue interface 3030 of energy transceiver 3010 may be operable to output one or more different energies 32 in signal direction SD toward the skin; and communication step 4160 may comprise selecting, with processing unit 3060, a combination of one or more different energies 32 based on a particular energy signal 90. As a further example, tissue interface 3030 may comprise a plurality of energy generators 31; each energy generator 31 may be operable to output a plurality of different energy types in the signal direction toward skin 1 (e.g., as shown in FIGS. 4A-D); and communication step 4160 may comprise: (i) selecting, with processing unit 3060, the combination of one or more different energies 32 and a group of generators from plurality of energy generators 31; and (ii) causing, with processing unit 3060, the selected group of energy generators 31 to output energy signal 90 with the combination of one or more different energies 32.

In keeping with above, each energy generator 31 may comprise a plurality of generator elements and each generator element may be operable to output one energy type of the plurality of different energies 32. As before, each energy generator of the plurality of generator elements may comprise one or more of: an impact generator element like element 36 of FIG. 3A; a thermal generator element like element 42 of FIG. 3B; an electrical stimulus generator element like element 48 of FIG. 3C; and a pressure generator element like element 52 of FIG. 3D.

An intensity of energy signal 90 may be varied in communicating step 4160 relative to the differential determined in step 4140 so as to provide user 1 with an identifiable sense of urgency prior to taking the associated corrective actions and an indicator of progress after taking the correction actions. For example, step 4160 may comprise: (i) causing, with processing unit 3060, energy transceiver 3010 to output energy signal 90 with the one or more different energies 32 at a minimum intensity when the differential is within a minimum range indicating that the actual physiological characteristics are consistent with the target physiological characteristics; and (ii) causing, with processing unit 3060, energy transceiver 3010 to output the energy signal with one or more different energies 32 at a maximum intensity when the differential is within a maximum range indicating that the actual physiological characteristics are not consistent with the target physiological characteristics. In this example, energy signal 90 may comprise a generally non-visual and/or non-audible combination of energies 32 that is minimally perceivable by the nerves associated with the physiologic tissue (e.g., skin 2) when output in communication step 4160 at a minimum intensity and maximally perceivable by the nerves associated with the physiologic tissue when output in step 4160 at a maximum intensity, making energy signal 90 somewhat ignorable when output at the minimal intensity and downright unavoidable when output at the maximal intensity.

Different types of energy signals 90 may be output responsive to the differential. For example, step 4160 also may comprise: (i) causing, with processing unit 3060, energy transceiver 3010 to output energy signal 90 with a first combination of the one or more different energies 32 when the differential is within the minimum range; and (ii) causing, with processing unit 3060, energy transceiver 3010 to output the energy signal with a second combination of the one or more different energies 32 when the differential is within the maximum range. In these examples, the energy signal may thus be continuously modified by processing unit 3060 during the second time period to guide user 1 toward the target physiological state by providing them with real-time feedback about the effectiveness of the corrective actions.

Communication step 4160 also may comprise causing, with processing unit 3060, a display device to output the guiding stimulus selected in step 4150 (if any). For example, step 4160 may comprise causing, with processing unit 3060, optical interface 3030′ to output a guiding stimulus that corresponds with the energy signal and comprises instructions for taking the correction. The guiding stimulus may comprise a video (e.g., one that is stored on YouTube® and accessible to processing unit 3060 over a network connection) containing a plurality of different stimulus types, each of which may help user 1 to reduce the differential by providing additional guidance thereto. For example, the plurality of different stimulus types may comprise any combination of an audible stimulus (e.g., spoken word) and/or a visual stimulus that corresponds with and is complementary to the energy signal, thereby providing user 1 with multiple different triggers in multiple different forms, further increasing the likelihood of compliance. Like the energy signal, the guiding stimulus also may be responsive to the differential and thus operable to provide real-time feedback. For example, the guiding stimulus may comprise a graphical representation of any change in the differential caused by the correction action (e.g., like a tachometer) and/or similarly vary an intensity level of optical interface 3030′ so that it, much like the energy signal, illuminates or dims responsive to the differential.

Method 4100 of enhancing a performance of user 1 also may comprise additional monitoring steps. For example, method 4100 also may comprise: (i) continuously monitoring, with processing unit 3060, the physiological signals of user 1 during the time period with physiological sensor 4012; (ii) determining, with processing unit 3060, the differential at different intervals during the time period; and (iii) automatically initiating, with processing unit 3060, the second time period by causing energy transceiver 3010 to output energy signal 90 (i.e., the trigger) when the differential for a preceding interval of the different intervals is greater than a minimum trigger value. In this example, system 4000 may continuously monitor physiological data associated with user 1 and automatically start outputting energy signal 90 to the physiologic tissue (e.g., skin 2) when the correction actions are required.

Additional monitoring may be performed in method 4100 to automatically terminate energy signal 90 after the correction actions have be successful performed so that energy signal 90 does not affect user 1 negatively after the differential has been reduced. For example, method 4100 may comprise: (i) continuously monitoring, with processing unit 3060, the physiological signals of user 1 during the second time period with physiological sensor 4012; (ii) determining, with processing unit 3060, the differential at different intervals during the second time period; and (iii) causing energy transceiver 3010 to cease outputting energy signal 90 to the physiologic tissue (e.g., skin 2) when the differential for a preceding interval of the second different intervals is less than a minimum trigger value for a minimum amount of time.

Steps 4110 to 4160 of method 4100, and any intermediate and/or additional steps related thereto, may be performed by one or more processors to enhance a performance of user 1 by causing the outputs of a trigger such as a particular energy signal 90 (e.g., an trigger) that has been previously associated with a particular corrective action (e.g., a breathing exercise) at times when the physiological data of user 1 suggests that the particular corrective action is necessary and/or required (e.g., when most likely experience anxiety). The triggers described with reference to method 4100 may thus compel user 1 to take the particular corrective action by utilizing outputs of energy signal 90 to communicate needs about taking corrective action and progress relating to the corrective action. In this regard, system 4000 and method 4100 may help user 1 to more consistently transition toward targeted physiological states by modifying the intensity and/or form of energy signal 90 (i.e., the trigger) relative to the differential so as to nudge user 1, with increasing intensity, toward taking corrective actions.

Additional training methods may be performed to support and/or increase the effectiveness of method 4100. An exemplary training method 4200 is now described with reference to system 4000 as comprising steps for generating a labelled data set by linking, with processing unit 3060, different frequencies or patterns of the plurality the physiological signals for user 1 with different confirmed physiological states for user 1. For example, training method 4200 may comprise: outputting to the eyes of user 1, with processing unit 3060 (e.g., via optical interface 3030′), a training stimulus adapted to induce a target physiological state for user 1 during a training period (an “outputting step 4210”); sensing, with processing unit 3060, a plurality of physiological signals of user 1 during the training period with physiological sensor 4012 (a “sensing step 4220”); determining, with processing unit 3060, based on the plurality of physiological signals sensed by physiological sensor 4012, physiological characteristics indicative of the target physiological state of user 1 when exposed to the training stimulus (a “determining step 4230”); receiving, with processing unit 3060 (e.g., via optical interface 3030′), inputs from user 1 confirming that they experienced the target physiological state during the training period (a “receiving step 4240”); establishing, with processing unit 3060, a link between the physiological signals and the confirmed target physiological state (an “establishing step 4250”); outputting to the eyes of user 1, with processing unit 3060 (e.g., via optical interface 3030′), a reduced form of the training stimulus during a second training period (an “reduced outputting step 4260”); and repeating steps 4230, 4240, and 4250 with each iteration of step 4260 to confirm that the target physiological state may be induced with the reduced form of the training stimulus (a “repeating step 4270).

Outputting step 4210 may vary according to training stimulus. As described herein, the training stimulus may comprise a plurality of different stimulus types including any combination of an audible signal (e.g., spoken word), visual signal (e.g., alphanumeric text), and/or an energy signal (e.g., as described herein) adapted to invoke a physiological response from user 1 during a training period. To provide a particular example, the training stimulus may comprise a video (e.g., one that is stored on YouTube® and accessible to processing unit 3060 over a network connection) output with optical interface 3030′ and/or another display device (e.g., a television that is not otherwise in data communication with processing unit 3060). The video may comprise music (or other background audio) containing a particular frequency or pattern adapted to invoke a particular physiological response, such as a binaural beat historically proven to transition user 1 from one physiological state (e.g., a low concentration brain state) to a target physiological state (e.g., a high concentration brain state). Multiple different types of stimulus may be output this way. For example, the video may comprise calming music containing a calming binaural beat together with a calming visual background containing calming textual instructions directing user 1 to complete a calming corrective action, such a breathing exercise.

Outputting step 4210 may comprise: selecting, with processing unit 3060, the energy signal based on the audio and/or visual signal; and causing, with processing unit 3060, energy transceiver 3010 to output the energy signal with together with the audio and/or visual signal. The energy signal may be selected manually or automatically. For example, step 4210 may comprise: (i) receiving, with processing unit 3060 (e.g., via optical interface 330′), a selection input from user 1 indicating a particular energy signal they would like to associate with the target mental state; and (ii) selecting, with processing unit 3060, the energy signal from a plurality of different energy signals based on the selection input. These steps may allow user 1 to select whatever combination of the one or more different energy types they are most comfortable with. For example, if other users have identified a certain energy signal as being a particular effective trigger (e.g., because of the particular combination of energies used therewith, then the certain energy signal may be made available to user 1 in step 4210 via an online store or similar means. In this example, a vast archive of proven energy signals may be compiled and made accessible to user 1.

The selection step of outputting step 4210 also may be performed automatically by: (i) identifying, with processing unit 3060, a frequency or pattern of the training stimulus; and (ii) selecting the energy signal based on the frequency or pattern. To continue the particular example from above, i.e., where the training stimulus comprises a YouTube video played by optical interface 3030′ and/or another display device, step 4210 also may comprise identifying, with processing unit 3060, the particular frequency or pattern contained in the video (e.g., the binaural beat_ that is identified by processing 3060 using sound analysis techniques (e.g., such as those employed by SoundHound® and similar technologies); and selecting, with processing unit 3060, an energy signal that complements and/or coincides with the particular frequency or pattern. This iteration of output step 4210 may be particular important when using a display device that is not in data communication with system 4000 because it allows the energy signal to be selected based on any existing content that includes the particular frequency or pattern, providing user 1 with access to a vast archive of potential training stimulus.

Sensing step 4220 and determining step 4230 of method 4200 may be performed in a manner similar to sensing step 4220 and determining step 4230 of method 4100 described above, with appropriate modifications for use in method 4200.

Receiving step 4240 may comprise receiving, with processing unit 3060 (e.g., via optical interface 330′), a selection input from user 1 indicating that they experienced the target physiological state during the training period. The selection may comprise a simple yes/or radio button and/or a more complex worksheet that helps to more precisely define their experience. For example, the selection may comprise a time bar indicating different intervals of the training period and user 1 may selection intervals during which they experienced the target physiological state so that the physiological signals output in step 4220 may be more closely linked to the target physiological state experienced during the selected interval.

Establishing step 4250 may comprise intermediate steps for creating a listing of energy signals and the target physiological states associated therewith. For example, the link may be defined by associating a particular frequency or pattern of the physiological data (e.g., as defined above) generated for user 1 during the training period with a particular combination of energies in the energy signal. As described herein, the listing may be utilized by user 1 at a later date to select a particular energy signal that they would like to use as a means for obtaining and/or avoiding a particular mental state with method 4100 described above. The listing may comprise a record of any training activities of user 1, including a measure of any spent forging an association between the energy signal and the target physiological state, thereby providing user 1 with an indicator regarding the likely effectiveness of each energy signal.

Reduced outputting step 4260 may be performed to help user 1 trigger the target mental state with simplified forms of the training stimulus until, after sufficient practice, they can reliably trigger the target physiological state using only the energy signal, even if they are in a high stimulus environment. This way, user 1 may utilize a complex training stimulus to forge an initial association between target physiological state and the energy signal, i.e., one that is reinforced by the audio and/or visual signals; and then progressively use different simplified forms of training stimulus to trigger the same association. In keeping with the particular example describe above, where the training stimulus is a YouTube video comprising calming music containing a calming binaural beat together with a calming visual background containing calming textual instructions, successive iterations of step 4260 may comprise outputting: a first reduced form that eliminates the calming background; a second reduced form that eliminates the calming music; a third reduced form that eliminates the calming textual instructions; and a fourth reduced form that eliminates the binaural beat, resulting in a training stimulus that consisting of nothing or than start and stop signals.

Repeating step 4270 may be performed with each iteration to 4260 to once again sense the physiological signals and determine the physiological state response to each reduced form of training stimulus so that user 1 may similarly confirm that method 4200 is working to help the associate the energy signal with the target physiological state using less and less training stimulus. For example, step 4270 may be performed numerous times by user 1 until they are confident that the target physiological stated may consistently triggered by the energy signal alone.

Method 4200 may thus provide a means for reliably triggering the target physiological state responsive to the energy signal by helping user 1 to forge a deep association between the energy signal and the target physiological state. As described above, the bond may be established with a complex training stimulus designed to invoke the target physiological state until it becomes a reflex response for user 1, and then reinforced over time using less and less stimulus until the energy signal, by itself, is sufficient to invoke the target physiological state. To provide an additional example, it may be much easier for user 1 to invoke the target physiological state responsive to the complex training signal, and that by itself may be valuable at times when user 1 can made ready use of a more immersive experience, such as when at home. Method 4200 may allow user 1 to similarly invoke the target physiological state outside of the home, in uncontrolled environments, with an energy signal that may not be noticeable to anyone else in that environment.

While principles of the present disclosure are disclosed herein with reference to illustrative aspects for particular applications, the disclosure is not limited thereto. Those having ordinary skill in the art and access to the teachings provided herein will recognize additional modifications, applications, aspects, and substitution of equivalents all fall in the scope of the aspects disclosed herein. Accordingly, the present disclosure is not to be considered as limited by the foregoing description.

1. A method of enhancing a performance of a user, the method comprising:
sensing, with a processing unit, a plurality of physiological signals of the user during a time period with one or more sensors proximate to the user;
determining, with the processing unit, physiological characteristics indicative of a physiological state of the user during the time period based on the plurality of physiological signals;
selecting, with the processing unit, target physiological characteristics indicative of a target physiological state of the user during a second time period;
determining, with the processing unit, a differential between the physiological characteristics and the target physiological characteristics;
selecting, with the processing unit, an energy signal associated with a corrective action performable by the user during the second time period to reduce the differential; and
communicating, with the processing unit, the energy signal to nerves associated with skin of the user during the second time period by causing an energy generator maintained against the skin to output the energy signal in a signal direction toward the skin with one or more different energy types at an intensity proportionate to the differential until the physiological characteristics are approximate to the target physiological characteristics.

2. The method of claim 1, wherein:
the plurality of physiological signals comprise brainwave signals; and
the one or more sensors comprise a brainwave sensor that is wearable by the user and adapted to output the brainwave signals responsive to activity of the user’s brain.

3. The method of claim 2, wherein the brainwave signals comprise measurements of electrical activity produced by the user’s brain.

4. The method of claim 2, wherein:
the plurality of physiological signals comprise heart signals; and
the one or more sensors comprise a heart sensor that is wearable by the user and adapted to output the heart signals responsive to activity of the user’s heart.

5. The method of claim 4, wherein the heart signals comprise measurements of electrical activity produced by the user’s heart.

6. The of method of claim 4, wherein:
the plurality of physiological signals comprise motion signals; and
the one or more sensors comprise a motion sensor that is wearable by the user and adapted to output the motion signals responsive to movements of the user’s body.

7. The method of claim 6, wherein the motion signals comprise measurements of electrical activity produced by the movements.

8. The method of claim 6, wherein:
the plurality of physiological signals comprise breath signals; and
the one or more sensors comprise a breath sensor that is wearable by the user and adapted to output the breath signals responsive to an activity of the user’s lungs.

9. The method of claim 8, wherein the breath signals comprise measurements of electrical activity produced by the user’s lungs.

10. The of method of claim 1, wherein determining the physiological characteristics comprises identifying a frequency or pattern of the plurality of physiological signals that corresponds to the physiological state.

11. The method of claim 10, wherein the selecting the target physiological characteristics comprises:
receiving, with the processing unit, a selection input from the user indicating the target physiological state; and
retrieving, with processing unit, the target physiological characteristics from a memory associated with the user based on the selection input received from the user.

12. The method of claim 11, wherein determining the differential comprises:
comparing, with the processing unit, the frequency or pattern corresponding to the physiological state with a target frequency or pattern corresponding to the target physiological state.

13. The method of claim 1, wherein selecting the energy signal comprises:
receiving, with the processing unit, the corrective action from a plurality of corrective actions based on one or more of:
the target physiological characteristics,
the differential, and
a criterion set by the user; and
selecting, with the processing unit, the energy signal from the plurality of different energy signals based on the received corrective action.

14. The method of claim 13, wherein:
the energy generator is operable to output a plurality of different energy types in the signal direction toward the skin; and
causing the energy generator to output the energy signal comprises:
selecting, with the processing unit, the one or more different energy types from the plurality of different energy types based on the energy signal.

15. The method of claim 1, wherein:
the energy generator comprises a plurality of energy generators, each energy generator of the plurality of energy generators is operable to output a plurality of different energy types in the signal direction toward the skin; and
causing the plurality of energy generators to output the energy signal comprises:
selecting, with the processing unit, the one or more different energy types from the plurality of different energy types and one or more energy generators of the plurality of energy generators; and
causing, with the processing unit, the one or more energy generators to output the energy signal using the one or more different energy types.

16. The method of claim 15, wherein each energy generator comprises a plurality of generator elements, and each generator element is operable to output one energy type of the plurality of different energy types in the signal direction.

17. The method of claim 16, wherein, for each energy generator, the plurality of generator elements comprises one or more of: an impact generator element; a heat generator element; a shock generator element; and a pressure generator element.

18. The method of claim 1, wherein communicating the energy signal comprises:
outputting the energy signal with the one or more different energy types at a minimum intensity when the differential is within a minimum range indicating that the physiological characteristics are consistent with the target physiological characteristics; and
outputting the energy signal with the one or more different energy types at a maximum intensity when the differential is within a maximum range indicating that the physiological characteristics are not consistent with the target physiological characteristics.

19. The method of claim 18, comprising:
outputting, with energy generator, the energy signal with a first combination of the one or more different energy types when the differential is within the minimum range; and
outputting, with the energy generator, the energy signal with a second combination of the one or more different energy types when the differential is within the maximum range.

20. The method of claim 1, comprising:
continuously monitoring, with the processing unit, the plurality of physiological signals of the user during the time period with the plurality of physiological sensors;
determining, with the processing unit, the differential at different intervals during the time period; and
automatically initiating, with the processing unit, the second time period by causing the energy generator to output the energy signal when the differential for a preceding interval of the different intervals is greater than a minimum trigger value.

21. The method of claim 20, comprising:
continuously monitoring, with the processing unit, the plurality of physiological signals of the user during the second time period with the plurality of physiological sensors;
determining, with the processing unit, the differential at different intervals during the second time period; and
causing the energy generator to cease outputting the energy signal when the differential for a preceding interval of the second different intervals is less than a minimum trigger value for a minimum amount of time.

22. The method of claim 1, wherein the target physiological state comprises one or more of:
brainwave signals indicating one of a high relaxation brain state, and a high concentration brain state;
heart signals indicating one of a low pulse rate, a low blood pressure, and a high blood oxygen level;
motion signals indicating one of a smooth motion rate, and a low impact motion rate; and
breath signals indicating one of a slow breathing rate, a depth of breath, and a high blood oxygen level.
